(19) United States
(12) Patent Application Publication
(10) Pub. No.: US 2026/0207585 A1
(43) Pub. Date: Jul. 23, 2026

(54) METHODS AND COMPOSITIONS FOR THE TREATMENT OF PARKINSON'S DISEASE, HUNTINGTON'S DISEASE, AMYOTROPHIC LATERAL SCLEROSIS, AND LEARNING DISORDERS

(71) Applicants: Cedars-Sinai Medical Center, Los Angeles, CA (US); Fortem Neurosciences, Inc., Sunny Isles Beach, FL (US)

(72) Inventors: Maya KORONYO, Los Angeles, CA (US); Keith L. BLACK, Los Angeles, CA (US); Jack KAVANAUGH, Los Angeles, CA (US); Larry COUTURE, Los Angeles, CA (US)

(21) Appl. No.: 19/140,195

(22) PCT Filed: Dec. 19, 2023
(86) PCT No.: PCT/US2023/084870

§ 371 Date: Jun. 17, 2025

Related U.S. Application Data
(60) Provisional application No. 63/476,331, filed on Dec. 20, 2022.
(60) Provisional application No. 63/504,860, filed on May 30, 2023.

(51) Int. Cl. A61K 31/4995

(52) U.S. Cl. CPC A61K 31/4995

(57) ABSTRACT

Provided are methods and compositions for the treatment of Parkinson's disease, Huntington's disease, amyotrophic lateral sclerosis, and learning disorders.

### CROSS-REFERENCE

This application claims the benefit of U.S. Provisional Application No. 63/476,331, filed on Dec. 20, 2022, and U.S. Provisional Application No. 63/504,860, filed on May 30, 2023, all of which are incorporated by reference herein.

### BACKGROUND

Parkinson's disease, Huntington's disease, amyotrophic lateral sclerosis and learning disorders all affect cognitive and behavioral processes. Treatment to ameliorate symptoms of these disorders are needed.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

There is an urgent unmet need to develop effective treatment for neurological diseases associated with synaptic and neuronal loss, detrimental neuroinflammation (e.g. microgliosis, astrogliosis) and increased H3K9me3 epigenome repression (e.g. associated with heterochromatin epigenetic silencing), for preserving neuromotor and cognitive functions. Hypermethylation of histones leads to gene silencing and reducing brain and synaptic plasticity, which may affect motor, cognitive, and visual functions. The methods and compositions described herein may involve inhibition or targeting of enzymes responsible for excessive histone methylation that may occur in the brain in neurodegenerative disorders. This may be advantageous in treating various neuromuscular and neurological disorders such as Parkinson's disease (PD), Huntington's disease (HD), and amyotrophic lateral sclerosis (ALS), leading to CNS-neuronal preservation and promoting new formation of neurons and synapses. Described herein are methods and compositions for reversing H3K9me3 epigenome gene repression and inducing transcription and translation of proteins that have critical functions in promoting neuronal survival and synaptic regeneration.

In certain aspects, described herein is a method of treating Parkinson's disease, Huntington's disease, ALS, or a learning disorder in a subject in need thereof, the method comprising administering to the subject a compound comprising an inhibitor of trimethylation of histone 3, lysine 9 (H3K9). In some embodiments, the compound comprises an inhibitor of SUV39H1. In some embodiments, the compound comprises an inhibitor of SUV39H2. In some embodiments, the compound has the structure of Formula (I), or a pharmaceutically acceptable salt thereof:

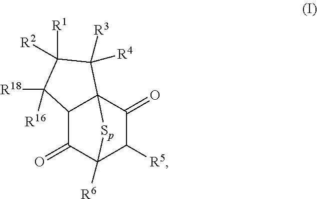

wherein, p is 2, 3 or 4, and
R¹, R², R³, R⁴, R⁵, R⁶, R¹⁶, and R¹⁸ are independently hydrogen, halogen, —N₃, —CF₃, —CCl₃, —CBr₃, —Cl₃, —CN, —CHO, —OH, —NH₂, —COOH, —CONH₂, —NO₂, —SH, —SO₂, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. In some embodiments, the compound comprises (Rac-(3S,6S,7S,8aS)-6-(benzo[d][1,3]dioxol-5-yl)-2,3,7-trimethyl-1,4-dioxohexahydro-6H-3, 8a-epidithiopyrrolo[1,2-a]pyrazine-7-carbonitrile) (ETP69), or a pharmaceutically acceptable salt thereof. In some embodiments, the administration improves locomotor activity in the subject. In some embodiments, the administration reduces neuroinflammation in the subject. In some embodiments, the administration increases or improves a neuroprotective phenotype. In some embodiments, the administration activates brain-derived neurotrophic factor (BDNF) or VGF nerve growth factor inducible (VGF) signaling in the subject. In some embodiments, the administration reduces or improves a phenotype associated with Parkinson's disease, Huntington's disease, ALS, or the learning disorder in the subject. In some embodiments, the subject has or has been diagnosed with Parkinson's disease. In some embodiments, the subject has or has been diagnosed with Huntington's disease. In some embodiments, the subject has or has been diagnosed with ALS. In some embodiments, the subject has or has been diagnosed with the learning disorder.

In certain aspects, described herein is a method of inhibiting an enzyme selected from the group consisting of: ASH1L, MLL1 complex, MLL4 complex, NSD3, Set1b complex, SMYD2, SMYD3, SUV39H1, and SUV39H2, comprising contacting the enzyme with a compound having the structure of Formula (I), or a pharmaceutically acceptable salt thereof:

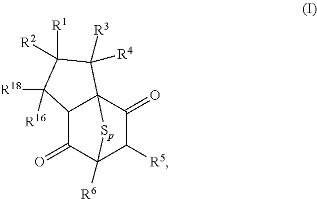

wherein, p is 2, 3 or 4, and
R¹, R², R³, R⁴, R⁵, R⁶, R¹⁶, and R¹⁸ are independently hydrogen, halogen, —N₃, —CF₃, —CCl₃, —CBr₃, —Cl₃, —CN, —CHO, —OH, —NH₂, —COOH, —CONH₂, —NO₂, —SH, —SO₂, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. In some embodiments, the compound comprises (Rac-(3S,6S,7S,8aS)-6-(benzo[d][1,3]dioxol-5-yl)-2,3,7-trimethyl-1,4-dioxohexahydro-6H-3, 8a-epidithiopyrrolo[1,2-a]pyrazine-7-carbonitrile) (ETP69), or a pharmaceutically acceptable salt thereof. In some embodiments, the contact is in vitro. In some embodiments, the contact is in vivo. In some embodiments, the contact is in a neuron of a subject. In some embodiments, the contact is in a human subject. In some embodiments, the method further comprises assessing an activity of the enzyme during or after the contact of the enzyme with the compound. In some embodiments, the activity of the enzyme is decreased by at least 75% after the contact of the enzyme with the compound.

### BRIEF DESCRIPTION OF THE DRAWINGS

FIG. 1A depicts Golgi-Cox staining in the cingulate cortex (cc) of mice treated with DMSO (top panels) and ETP69 (lower panels).

FIG. 1B depicts Golgi-Cox staining in the hippocampus.

FIG. 1C depicts individual neurons of DMSO-treated and ETP69-treated neurons.

FIG. 1D depicts a schematic of the different types of dendritic spines quantified.

FIG. 1E depicts the quantification of thin spines in the cingulate cortex and the hippocampus.

FIG. 1F depicts correlations between the number of thin spines in the hippocampus and the number of errors and seconds freezing.

FIG. 1G depicts the quantification of dendritic spines in the cingulate cortex and the hippocampus.

FIG. 1H depicts correlations between the ratio of mushroom to dendritic spines in the hippocampus and the number of errors and seconds freezing.

FIG. 2A depicts the experimental configuration.

FIG. 2B depicts the regions of the brain isolated for further analysis.

FIG. 2C depicts H3K9me3 staining in the layers of the cingulate cortex.

FIG. 2D depicts H3K9me3 staining in DMSO-treated (upper panel) and ETP69-treated (lower panel) brains.

FIG. 2E depicts the correlation between the percent of alternations in the test and H3K9me3 irradiance.

FIG. 2F depicts a blot showing H3K9me3 in DMSO and ETP69 treated AD+ mice.

FIG. 2G depicts H3K9me3 staining in DMSO and ETP69 treated brains.

FIG. 2H depicts a quantification of H3K9me3 irradiance.

FIG. 2I depicts representative images of H3K9me3 immunofluorescence.

FIG. 2J depicts a quantification of H2K9me3 in the cingulate cortex of DMSO-treated and ETP69-treated animals.

FIG. 2K depicts levels of H3K9me3 levels normalized to actin levels in mice treated with DMSO and ETP69.

FIG. 2L depicts a quantification of H2K9me3 in the hippocampus of DMSO-treated and ETP69-treated animals.

FIG. 2M depicts the correlations between H3K9me3 staining and the number of errors or the seconds freezing.

FIG. 3 depicts the genes activated in the BDNF and VGF protective pathways.

FIG. 4A depicts genes activated and inactivated by ETP69.

FIG. 4B depicts quantification of PLTP in DMSO-treated and ETP69-treated mice.

FIG. 4C depicts quantification of Annexin 2 in DMSO-treated and ETP69-treated mice.

FIG. 4D depicts Annexin 2 and PLTP staining in DMSO-treated and ETP69-treated mice.

FIG. 4E depicts a quantification of PLTP irradiance in DMSO-treated and ETP69-treated mice.

FIG. 4F depicts a quantification of Annexin 2 irradiance in DMSO-treated and ETP69-treated mice.

FIG. 4G depicts a quantification of BDNF irradiance. In DMSO and ETP69+AD+ mice.

FIG. 4H depicts a quantification of VGF irradiance in the hippocampus of DMSO-treated and ETP69-treated mice.

FIG. 4I depicts BDNF, VGF, and Thio-S staining in DMSO-treated and ETP69-treated mice.

FIG. 4J depicts a quantification of TTC3 in DMSO-treated and ETP69-treated mice (left panel). TRKB in DMSO-treated and ETP69-treated mice (center panel) and VGF in DMSO-treated and ETP69-treated mice (right panel).

FIG. 4K depicts a correlation between VGF irradiance and H3K9me3 irradiance in the hippocampus.

FIG. 4L depicts a quantification of VGF irradiance in the cingulate cortex of DMSO-treated and ETP69-treated mice.

FIG. 4M depicts a correlation between the number of errors at day 2 and the levels of VGF irradiance in the cingulate cortex.

FIG. 5 depicts the experimental protocol used to test the effects of ETP69 on mice.

FIG. 6A depicts numbers of total entries into the Y maze.

FIG. 6B depicts percent of alternations in the Y maze.

FIG. 6C depicts a schematic of the visual stimuli X maze and the number of total entries into the visual stimuli X maze.

FIG. 6D depicts percent of alternations in the visual stimuli X maze.

FIG. 6E depicts percent transitions in the visual stimuli X maze.

FIG. 6F depicts percent of alternations in the visual stimuli X maze.

FIG. 6G depicts a schematic of the high-contrast visual stimuli X maze as well as the total number of entries.

FIG. 6H depicts percent of alternations in the high contrast visual stimuli X maze.

FIG. 7A depicts a schematic of the Barnes maze.

FIG. 7B depicts the number of errors in the Barnes Maze of mice administered ETP69 or DMSO during training at day 10.

FIG. 7C depicts the number of errors in the reversal phase of the Barnes Maze Test.

FIG. 7D depicts a schematic of the fear conditioning test.

FIG. 7E depicts the results of the fear conditioning test.

FIG. 8A depicts the representative images of AD+ mice brains stained with H3K9me3 and DAPI.

FIG. 8B depicts the relationship between levels of H3K9me3 irradiance in the cingulate cortex and different treatment conditions at 5 days post treatment (left panel) and 15 days post treatment (right panel).

FIG. 8C depicts H3K9me3 and DAPI staining in the hippocampus of AD+ mice administered DMSO or ETP69.

FIG. 8D depicts the levels of H3K9me3 irradiance in the hippocampus of mice administered DMSO and ETP69.

FIG. 8E depicts the relationship between the number of errors in the reversal phase of the Barnes test with H3K9me3 irradiance at day 15 with DG and the relationship between the seconds to freezing in the fear conditioning test and H3K9me3 irradiance at day 15.

FIG. 9A depicts representative images of H3K9me2, 6E10, and GFAP staining in mice administered DMSO and ETP69.

FIG. 9B depicts a quantification of GFAP staining in the cingulate cortex of mice administered DMSO and ETP69 4 days (left panel) and 15 days (right panel) after treatment.

FIG. 9C depicts a quantification of 6E10 staining in the cingulate cortex of mice administered DMSO and ETP69 4 days (left panel) and 15 days (right panel) after treatment.

FIG. 10A depicts a representative image of a Golgi-Cox stained cingulate cortex of a mouse administered DMSO.

FIG. 10B depicts a representative image of a Golgi-Cox stained hippocampus of a mouse administered DMSO.

FIG. 10C depicts a representative image of a Golgi-Cox stained neuron of a mouse administered DMSO.

FIG. 10D depicts a representative image of a Golgi-Cox stained cingulate cortex of a mouse administered ETP69.

FIG. 10E depicts a representative image of a Golgi-Cox stained hippocampus of a mouse administered ETP69.

FIG. 10F depicts a representative image of a Golgi-Cox stained neuron of a mouse administered ETP69.

FIG. 10G depicts quantification of dendritic spines in the cingulate cortex and hippocampus.

FIG. 10H depicts quantification of PSD95 irradiance.

FIG. 11A depicts representative images of H3K9me3, NeuN and DAPI staining in AD+ mice administered DMSO or ETP69.

FIG. 11B depicts a quantification of neurons in mice administered DMSO or ETP69.

FIG. 11C depicts representative images of neurons stained with H3K9 and GFAP in AD+ mice administered DMSO or ETP69.

FIG. 11D depicts a quantification of neurons stained with H3K9 and GFAP in AD+ mice administered DMSO or ETP69.

FIG. 11E depicts representative images of neurons stained with H3K9, CD45, Ilba-1, 6E10, and DAPI in AD+ mice administered DMSO or ETP69.

FIG. 11F depicts a quantification of microglia macrophages in AD+ mice administered DMSO or ETP69.

FIG. 12A depicts a principle component analysis of the proteomic data.

FIG. 12B depicts a plot showing the inverse relationship of the FC in the protein expression of the overlapping significant DEPs in AD+ mice (versus WT mice) and ETP69-treated AD+ mice (versus DMSO-treated AD+ mice).

FIG. 12C depicts a volcano plot comparing protein levels of ETP69 and DMSO-treated mice.

FIG. 12D depicts a pathway analysis of proteomic data.

FIG. 13A depicts genes inactivated and activated by ETP69 administration.

FIG. 13B depicts representative images BDNF, VGF, and Thio-S in mice administered DMSO and ETP69.

FIG. 13C depicts representative images of immunofluorescence of BDNF, VGF, and Thio-S in mice administered ETP69 or DMSO.

FIG. 13D depicts a quantification of BDNF in mice administered ETP69.

FIG. 13E depicts a quantification of VGF in mice administered ETP69.

FIG. 13F depicts the relationship between levels of VGF and H3K9me3 staining in the hippocampus.

FIG. 13G depicts the relationship between VGF expression and the number of errors in the reversal phase of the test.

FIG. 14 depicts a chart of differentially expressed proteins between ETP69 and DMSO.

FIG. 15A depicts the percentage of alternations in the colour mode of the ViS4M test. #p<0.05, ##p<0.01, ###p<0.001, and ####p<0.0001: DMSO-treated WT mice versus DMSO-treated AD⁺ mice; ★p<0.05, ★★p<0.01, ★★★p<0.001, and ★★★★p<0.0001: DMSO-treated mice versus ETP69-treated mice; one-way ANOVA followed by Fisher's LSD post hoc test or unpaired Student's t test.

FIG. 15B depicts the density of dendritic spines as counts per 100 m of dendrite. #p<0.05, ##p<0.01, ###p<0.001, and ####p<0.0001: DMSO-treated WT mice versus DMSO-treated AD⁺ mice; ★p<0.05, ★★p<0.01, ★★★p<0.001, and ★★★★p<0.0001: DMSO-treated mice versus ETP69-treated mice; one-way ANOVA followed by Fisher's LSD post hoc test or unpaired Student's t test.

FIG. 15C depicts Pearson's coefficient between the cortical and hippocampal thin spine density and behavioral performance in the Barnes maze and the context-specific fear conditioning tests.

FIG. 15D depicts the ratio of thin spines to total spines and ratio of mushroom spines to total spines. #p<0.05, ##p<0.01, ###p<0.001, and ####p<0.0001: DMSO-treated WT mice versus DMSO-treated AD+ mice; ★p<0.05, ★★p<0.01, ★★★p<0.001, and ★★★★p<0.0001: DMSO-treated mice versus ETP69-treated mice; one-way ANOVA followed by Fisher's LSD post hoc test or unpaired Student's t test.

FIG. 15E depicts the Pearson's correlation between the cortical thin spine and hippocampal mushroom spine ratios and performance in the retention phase of the Barnes maze test.

FIG. 16A depicts a representative mage of H3K9me3 IR in the cortex showing areas used for measurements (layers II/III and VI) and quantification of cortical H3K9me3 IR. #p<0.05, ##p<0.01, ###p<0.001, and ####p<0.0001: DMSO-treated WT mice versus DMSO-treated AD+ mice; ★p<0.05, ★★p<0.01, ★★★p<0.001, and ★★★★p<0.0001: DMSO-treated mice versus ETP69-treated mice; one-way ANOVA followed by Fisher's LSD post hoc test or unpaired Student's t test.

FIG. 16B depicts Pearson's coefficient between cortical H3K9me3 IR and performance in colour mode in the ViS4M test.

FIG. 16C depicts representative images of H3K9me3 IR in the hippocampus showing the cornu ammonis (CA) and the dentate gyrus (DG) areas used for measurements and corresponding quantifications of H3K9me3 IR. #p<0.05, ##p<0.01, ###p<0.001, and ####p<0.0001: DMSO-treated WT mice versus DMSO-treated AD+ mice; ★p<0.05, ★★p<0.01, ★★★p<0.001, and ★★★★p<0.0001: DMSO-treated mice versus ETP69-treated mice; one-way ANOVA followed by Fisher's LSD post hoc test or unpaired Student's t test.

FIG. 16D depicts a quantification for Iba-1 IR (red; indicating activated microglia) in the cerebral cortex. Group means, SEMs and individual data points are shown. p<0.05, ★★p<0.01, ★★★p<0.001, and ★★★★p<0.0001: DMSO-treated mice versus ETP69-treated mice; one-way ANOVA followed by Fisher's LSD post hoc test or unpaired Student's t test.

FIG. 17A depicts the inverse relationship of the FC in the protein expression of the 162 overlapping significant DEPs in AD+ mice (versus WT mice) and ETP69-treated AD⁺ mice (versus DMSO-treated AD⁺ mice). In cluster 1, the expression of 89 proteins was upregulated in AD+ mice and downregulated after ETP69 treatment. In cluster 2, the expression of 70 proteins was downregulated in ADX mice and upregulated after ETP69 treatment.

FIG. 17B depicts heatmaps comparing activation z scores for biological processes associated with behaviour and with neuroplasticity in AD+ mice (versus WT mice) and ETP69-treated AD+ mice (versus DMSO-treated AD+ mice) determined by IPA (Qiagen).

FIG. 17C depicts STRING v11.5 protein association network showing overlapping proteins involved in ‘learning’, ‘quantity of neurons’ and ‘dendritic growth/branching’ according to IPA. Red nodes: upregulated. Green nodes: downregulated. Edge thickness: from medium (0.7)- to high-confidence (0.9) protein association.

FIG. 17D depicts heatmaps comparing IPA activation z scores for upstream regulators in AD+ mice (versus WT mice) and ETP69-treated AD+ mice (versus DMSO-treated AD+ mice).

### DETAILED DESCRIPTION

There is an urgent unmet need to develop effective treatment for neurological diseases that may be commonly associated with synaptic and neuronal loss, detrimental neuroinflammation (e.g. microgliosis, astrogliosis) and increased H3K9me3 epigenome repression (e.g. associated with heterochromatin epigenetic silencing), for preserving neuromotor and cognitive functions. Described herein are methods of reversing H3K9me3 epigenome gene repression and inducing transcription and translation of proteins that may have important functions in promoting neuronal survival and synaptic regeneration. Without being limited by theory, the inhibition of enzyme(s) responsible for excessive histone methylation that may occur in the brain with aging and in neurodegenerative disorders. Hypermethylation of histones may lead to gene silencing and reduces brain and synaptic plasticity, which may be important for motor, cognitive, and visual functions.

In some embodiments, described herein is the use of epipolythiodioxopiperazines (ETPs) as ETP69 or analogs (or any histone tri-methylation inhibitor) to treat Parkinson's disease (PD), Huntington's disease (HD), amyotrophic lateral sclerosis (ALS), and other neurological disorders. In some embodiments, described herein is the use of histone methyltransferase SUV39H1 inhibitors to treat Parkinson's disease (PD), Huntington's disease (HD), amyotrophic lateral sclerosis (ALS), and other neurological disorders.

In the Examples described herein, ETP69 administration in old and middle aged wildtype (WT) and neurodegenerative mice protected against or reversed locomotor, cognitive, and behavioral dysfunctions. Along with substantially enhancing neuronal dendrite formation (especially the newly-formed thin dendrites) and synaptic markers, ETP69 activated brain proteins related to neurotrophic support, immune regulation and homeostatic balance. Neurotrophic factors (NTFs) are molecules that enhance growth and survival of neurons. Indeed, ETP69 activated BDNF, increased VGF nerve inducible factor and levodopa (dopaminergic neurons) levels in the brain. In addition to activating BDNF, the data described herein includes substantial increases in cortical and hippocampal VGF nerve factor and other factors by ETP69 that can preserve neuronal structure and function and their connections (synapses). The delivery efficacy and poor diffusion of some NTFs through the blood-brain barrier and inside the brain are considered the major problems behind their modest effects in clinical trials. ETP69 was found to have a good delivery to the brain and it causes a major increase in NTFs.

In some embodiments, the methods and compositions described herein provide an advantage in treating various neuromuscular and neurological disorders such as Parkinson's disease (PD), Huntington's disease (HD), and amyotrophic lateral sclerosis (ALS). Administration of these methods may lead to CNS-neuronal preservation and promoting new formation of neurons and synapses. Further, without being limited by theory, ETP69 showed protective effects on microglia and astrocytes—the CNS's support cells that are important for repair, maintaining tissue homeostasis, and/or phagocytosis of abnormal proteins/cancer cells/pathogens.

In some embodiments, administration of ETP69 may comprise a disease-modifying therapy. Without being limited by theory, administration of ETP69 may result not only in reversing the biological ageing clock of neurons and supplying the central nervous system with neurotrophic support, but may also rejuvenate the non-neuronal glial cells. Administration of ETP69 may preserve neuromuscular and cognitive-behavioral functions.

Methylation of a histone tail typically occurs at specific lysine residues, such as H3K4, H3K9, H3K27, H3K36, H3K79 and H4K20, and may activate or repress transcription. H3K9me3 may be a repressive histone mark, and is typically implicated in gene silencing. Some embodiments include a role for histone H3K9me3 and its histone methyl transferase (SUV39H1) in mediating hippocampal memory functions, and affecting Parkinson's disease, Huntington's disease, amyotrophic lateral sclerosis (ALS), or a learning disorder progression or development. Pharmacological inhibition of SUV39H1 using a selective inhibitor may decrease levels of H3K9me3 in the hippocampus of treated subjects, and/or improve performance in an object location memory task, a fear conditioning task or a complex spatial environment learning task. The inhibition of SUV39H1 by ETP69 or another compound disclosed herein may induce an increase in spine density of thin and stubby but not mushroom spines in the hippocampus of a treated subject, and increase GluR1-containing AMPA receptors levels at spine surface, a useful index of long-term potentiation (LTP). Establishment of H3K9me3 may depend on activity of the histone methyl transferase SUV39H1 which regulates H3K9 trimethylation at the peri-centric heterochromatin. Regulating the function of enzymes that contribute to histone methylation may hence be a powerful means to offset cognitive deficits.

In some embodiments, described herein are methods of treating cognitive dysfunction by inhibiting an enzyme selected from the group consisting of ASH1L, MLL1 complex, MLL4 complex, NSD3, Set1b complex, SMYD2, SMYD3, SUV39H1, SUV39H2, G9a, MLL2 Complex, MLL3 Complex, NSD1, NSD2, NSD2 (E1099K), NSD2 (T1150A), PRDM9, PRMT7, SETDB1, and SETD2. In some embodiments, described herein are methods of treating cognitive dysfunction by inhibiting a histone methyltransferase. In some embodiments, the histone methyltransferase comprises SUV39H1. In some embodiments, described herein are methods of treating cognitive dysfunction by inhibiting a histone methyltransferase other than SUV39H1. In some embodiments, the cognitive dysfunction comprises Parkinson's disease, Huntington's disease, ALS, or a learning disorder.

### A. COMPOUNDS

Provided herein, are compounds for use in methods of treating, preventing or delaying onset of Parkinson's disease, Huntington's disease, amyotrophic lateral sclerosis (ALS), or a learning disorder. In some embodiments, the compound inhibits a histone methyl transferase. In some embodiments, the compound inhibits SUV39H1. In some embodiments, the compound inhibits trimethylation of H3K9 (H3K9me3). For example, some embodiments include use of H3K9me3 modulation for enhancing cognitive function or treatment of disorders such as Parkinson's disease, Huntington's disease, ALS, or a learning disorder.

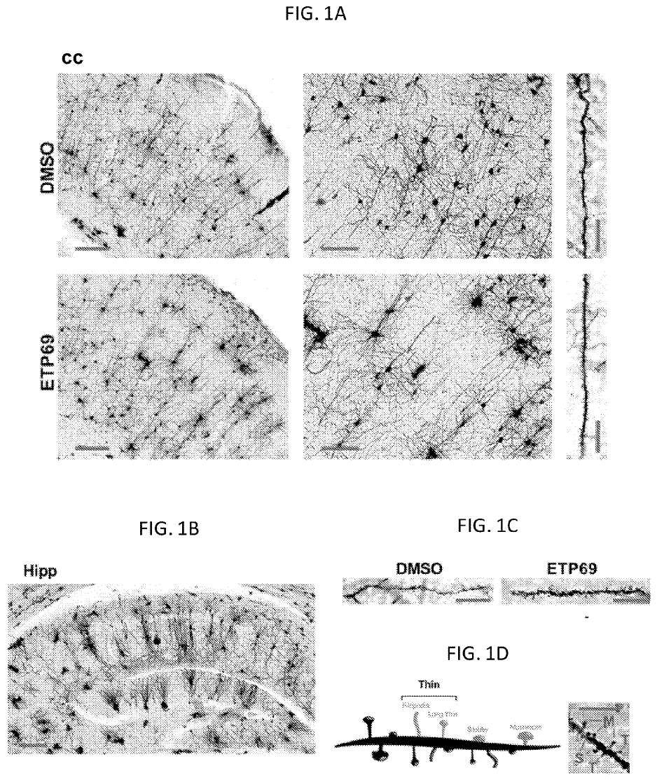

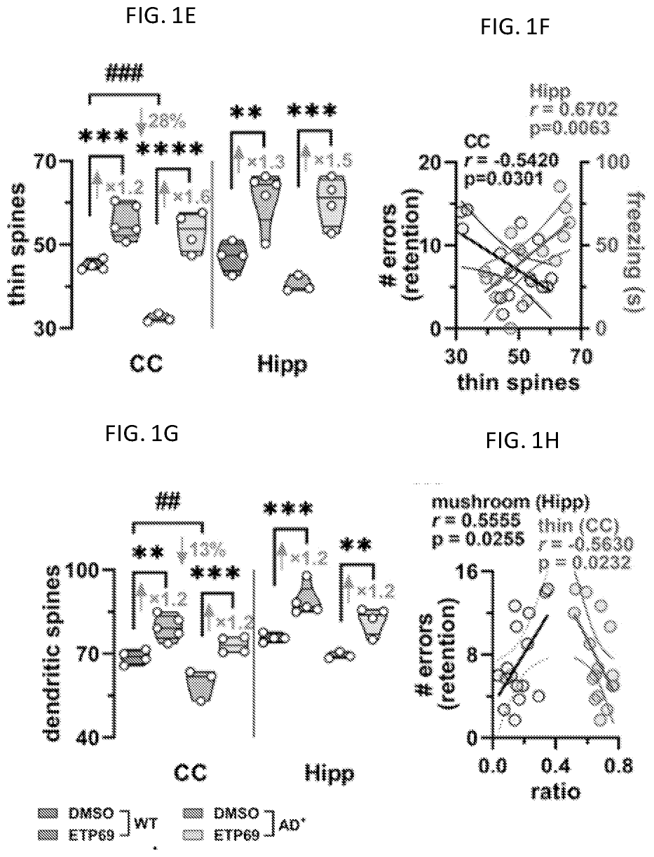

In some embodiments, the compound comprises ETP69 (Rac-(3S,6S,7S,8aS)-6-(benzo[d][1,3]dioxol-5-yl)-2,3,7-trimethyl-1,4-dioxohexahydro-6H-3,8a-epidithiopyrrolo[1,2-a]pyrazine-7-carbonitrile). In some embodiments, the compound consists of ETP69. A structure of ETP69 is shown in FIG. 1. In some embodiments, the compound comprises ETP69, or a pharmaceutically acceptable salt thereof. In some embodiments, the compound consists of ETP69, or a pharmaceutically acceptable salt thereof.

In some embodiments, the compound includes an analog of ETP69. In some embodiments, the compound is an analog of ETP69. In some embodiments, the compound comprises or consists of an analog of ETP69, or a pharmaceutically acceptable salt thereof. In some embodiments, the analog of ETP69 is a compound having the following formula:

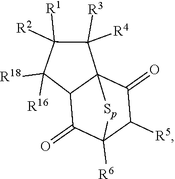

The symbol p may be 2, 3 or 4. In some embodiments, p is 2. In some embodiments, p is 3. In some embodiments, p is 4.

R¹, R², R³, R⁴, R⁵, R⁶, R¹⁶, and/or R¹⁸ may each independently be hydrogen, a halogen, —N₃, —CF₃, —CCl₃, —CBr₃, —Cl₃, —CN, —CHO, —OH, —NH₂, —COH, —CONH₂, —NO₂, —SH, —SO₂, —SO₂Cl, —SO₃H, —SO₄H, —SO₂NH₂, —NHNH₂, —ONH₂, —NHC(O)NHNH₂, a substituted or unsubstituted alkyl, a substituted or unsubstituted heteroalkyl, a substituted or unsubstituted cycloalkyl, a substituted or unsubstituted heterocycloalkyl, a substituted or unsubstituted aryl, or a substituted or unsubstituted heteroaryl.

In some embodiments, R¹ is hydrogen, a halogen, —N₃, —CF₃, —CCl₃, —CBr₃, —Cl₃, —CN, —CHO, —OH, —NH₂, —COH, —CONH₂, —NO₂, —SH, —SO₂, —SO₂Cl, —SO₃H, —SO₄H, —SO₂NH₂, —NHNH₂, —ONH₂, —NHC(O)NHNH₂, a substituted or unsubstituted alkyl, a substituted or unsubstituted heteroalkyl, a substituted or unsubstituted cycloalkyl, a substituted or unsubstituted heterocycloalkyl, a substituted or unsubstituted aryl, or a substituted or unsubstituted heteroaryl.

In some embodiments, R² is hydrogen, a halogen, —N₃, —CF₃, —CCl₃, —CBr₃, —Cl₃, —CN, —CHO, —OH, —NH₂, —COOH, —CONH₂, —NO₂, —SH, —SO₂, —SO₂Cl, —SO₃H, —SO₄H, —SO₂—NH₂, —NHNH₂, —ONH₂, —NHC(O)NHNH₂, a substituted or unsubstituted alkyl, a substituted or unsubstituted heteroalkyl, a substituted or unsubstituted cycloalkyl, a substituted or unsubstituted heterocycloalkyl, a substituted or unsubstituted aryl, or a substituted or unsubstituted heteroaryl.

In some embodiments, R³ is hydrogen, a halogen, —N₃, —CF₃, —CCl₃, —CBr₃, —Cl₃, —CN, —CHO, —OH, —NH₂, —COH, —CONH₂, —NO₂, —SH, —SO₂, —SO₂Cl, —SO₃H, —SO₄H, —SO₂—NH₂, —NHNH₂, —ONH₂, —NHC(O)NHNH₂, a substituted or unsubstituted alkyl, a substituted or unsubstituted heteroalkyl, a substituted or unsubstituted cycloalkyl, a substituted or unsubstituted heterocycloalkyl, a substituted or unsubstituted aryl, or a substituted or unsubstituted heteroaryl.

In some embodiments, R⁴ is hydrogen, a halogen, —N₃, —CF₃, —CCl₃, —CBr₃, —Cl₃, —CN, —CHO, —OH, —NH₂, —COH, —CONH₂, —NO₂, —SH, —SO₂, —SO₂Cl, —SO₃H, —SO₄H, —SO₂—NH₂, —NHNH₂, —ONH₂, —NHC(O)NHNH₂, a substituted or unsubstituted alkyl, a substituted or unsubstituted heteroalkyl, a substituted or unsubstituted cycloalkyl, a substituted or unsubstituted heterocycloalkyl, a substituted or unsubstituted aryl, or a substituted or unsubstituted heteroaryl.

In some embodiments, R⁵ is hydrogen, a halogen, —N₃, —CF₃, —CCl₃, —CBr₃, —Cl₃, —CN, —CHO, —OH, —NH₂, —COOH, —CONH₂, —NO₂, —SH, —SO₂, —SO₂Cl, —SO₃H, —SO₄H, —SO₂NH₂, —NHNH₂, —ONH₂, —NHC(O)NHNH₂, a substituted or unsubstituted alkyl, a substituted or unsubstituted heteroalkyl, a substituted or unsubstituted cycloalkyl, a substituted or unsubstituted heterocycloalkyl, a substituted or unsubstituted aryl, or a substituted or unsubstituted heteroaryl.

In some embodiments, R⁶ is hydrogen, a halogen, —N₃, —CF₃, —CCl₃, —CBr₃, —Cl₃, —CN, —CHO, —OH, —NH₂, —COOH, —CONH₂, —NO₂, —SH, —SO₂, —SO₂Cl, —SO₃H, —SO₄H, —SO₂NH₂, —NHNH₂, —ONH₂, —NHC(O)NHNH₂, a substituted or unsubstituted alkyl, a substituted or unsubstituted heteroalkyl, a substituted or unsubstituted cycloalkyl, a substituted or unsubstituted heterocycloalkyl, a substituted or unsubstituted aryl, or a substituted or unsubstituted heteroaryl.

In some embodiments, R¹⁶ is hydrogen, a halogen, —N₃, —CF₃, —CCl₃, —CBr₃, —Cl₃, —CN, —CHO, —OH, —NH₂, —COOH, —CONH₂, —NO₂, —SH, —SO₂, —SO₂Cl, —SO₃H, —SO₄H, —SO₂NH₂, —NHNH₂, —ONH₂, —NHC(O)NHNH₂, a substituted or unsubstituted alkyl, a substituted or unsubstituted heteroalkyl, a substituted or unsubstituted cycloalkyl, a substituted or unsubstituted heterocycloalkyl, a substituted or unsubstituted aryl, or a substituted or unsubstituted heteroaryl.

In some embodiments, R¹⁸ is hydrogen, a halogen, —N₃, —CF₃, —CCl₃, —CBr₃, —Cl₃, —CN, —CHO, —OH, —NH₂, —COOH, —CONH₂, —NO₂, —SH, —SO₂, —SO₂Cl, —SO₃H, —SO₄H, —SO₂NH₂, —NHNH₂, —ONH₂, —NHC(O)NHNH₂, a substituted or unsubstituted alkyl, a substituted or unsubstituted heteroalkyl, a substituted or unsubstituted cycloalkyl, a substituted or unsubstituted heterocycloalkyl, a substituted or unsubstituted aryl, or a substituted or unsubstituted heteroaryl.

In some embodiments, R¹, R², R³, R⁴, R⁵, R⁶, R¹⁶, or R¹⁸ is hydrogen. In some embodiments, R¹, R², R³, R⁴, R⁵, R⁶, R¹⁶, or R¹⁸ is a halogen. In some embodiments, R¹, R², R³, R⁴, R⁵, R⁶, R¹⁶, or R¹⁸ is N₃. In some embodiments, R¹, R², R³, R⁴, R⁵, R⁶, R¹⁶, or R¹⁸ is —CF₃. In some embodiments, R¹, R², R³, R⁴, R⁵, R⁶, R¹⁶, or R¹⁸ is —CCl₃. In some embodiments, R¹, R², R³, R⁴, R⁵, R⁶, R¹⁶, or R¹⁸ is —CBr₃. In some embodiments, R¹, R², R³, R⁴, R⁵, R⁶, R¹⁶, or R¹⁸ is —Cl₃. In some embodiments, R¹, R², R³, R⁴, R⁵, R⁶, R¹⁶, or R¹⁸ is —CN. In some embodiments, R¹, R², R³, R⁴, R⁵, R⁶, R¹⁶, or R¹⁸ is —CHO. In some embodiments, R¹, R², R³, R⁴, R⁵, R⁶, R¹⁶, or R¹⁸ is —OH. In some embodiments, R¹, R², R³, R⁴, R⁵, R⁶, R¹⁶, or R¹⁸ is —NH₂. In some embodiments, R¹, R², R³, R⁴, R⁵, R⁶, R¹⁶, or R¹⁸ is —COH. In some embodiments, R¹, R², R³, R⁴, R⁵, R⁶, R¹⁶, or R¹⁸ is —CONH₂. In some embodiments, R¹, R², R³, R⁴, R⁵, R⁶, R¹⁶, or R¹⁸ is —NO₂. In some embodiments, R¹, R², R³, R⁴, R⁵, R⁶, R¹⁶, or R¹⁸ is —SH. In some embodiments, R¹, R², R³, R⁴, R⁵, R⁶, R⁶, or R¹⁸ is —SO₂. In some embodiments, R¹, R², R³, R⁴, R⁵, R⁶, R¹⁶, or R¹⁸ is —SO₂Cl. In some embodiments, R¹, R², R³, R⁴, R⁵, R⁶, R¹⁶, or R¹⁸ is —SO₃H. In some embodiments, R¹, R², R³, R⁴, R⁵, R⁶, R¹⁶, or R¹⁸ is —SO₄H. In some embodiments, R¹, R², R³, R⁴, R⁵, R⁶, R¹⁶, or R¹⁸ is —SO₂NH₂. In some embodiments, R¹, R², R³, R⁴, R⁵, R⁶, R¹⁶, or R¹⁸ is —NHNH₂. In some embodiments, R¹, R², R³, R⁴, R⁵, R⁶, R¹⁶, or R¹⁸ is —ONH₂. In some embodiments, R¹, R², R³, R⁴, R⁵, R⁶, R¹⁶, or R¹⁸ is —NHC(O)NHNH₂. In some embodiments, R¹, R², R³, R⁴, R⁵, R⁶, R¹⁶, or R¹⁸ is a substituted or unsubstituted alkyl. In some embodiments, R¹, R², R³, R⁴, R⁵, R⁶, R¹⁶, or R¹⁸ is a substituted or unsubstituted heteroalkyl. In some embodiments, R¹, R², R³, R⁴, R⁵, R⁶, R¹⁶, or R¹⁸ is a substituted or unsubstituted cycloalkyl. In some embodiments, R¹, R², R³, R⁴, R⁵, R⁶, R¹⁶, or R¹⁸ is a substituted or unsubstituted heterocycloalkyl. In some embodiments, R¹, R², R³, R⁴, R⁵, R⁶, R¹⁶, or R¹⁸ is a substituted or unsubstituted aryl. In some embodiments, R¹, R², R³, R⁴, R⁵, R⁶, R¹⁶, or R¹⁸ is a substituted or unsubstituted heteroaryl.

In some embodiments, the compound is a compound that inhibits a methyltransferase that is not SUV38H1, wherein the compound is not ETP69. In some embodiments, the compound is a siRNA. In some embodiments, the compound is a peptide. In some embodiments, the compound is an antibody. In some embodiments, the methyltransferase is selected from the group consisting of ASH1L, MLL1 complex, MLL4 complex, NSD3, Set1b complex, SMYD2, SMYD3, SUV39H2, G9a, MLL2 Complex, MLL3 Complex, NSD1, NSD2, NSD2 (E1099K), NSD2 (T1150A), PRDM9, PRMT7, SETDB1, and SETD2.

In some embodiments, the compound is used in the manufacture of a medicament for treatment of Parkinson's disease, Huntington's disease, ALS, or a learning disorder. In some embodiments, the compound is used in the manufacture of a medicament for prevention of Parkinson's disease, Huntington's disease, ALS, or a learning disorder. In some embodiments, the compound is used in the manufacture of a medicament for delaying onset of Parkinson's disease, Huntington's disease, ALS, or a learning disorder.

### B. FORMULATIONS

In certain embodiments, the compound as described herein is administered as a pure chemical. In other embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration.

In some embodiments, the composition is a pharmaceutical composition. In some embodiments, the composition is sterile. In some embodiments, the composition further comprises a pharmaceutically acceptable carrier.

In some embodiments, the pharmaceutically acceptable carrier comprises water. In some embodiments, the pharmaceutically acceptable carrier comprises a buffer. In some embodiments, the pharmaceutically acceptable carrier comprises a saline solution. In some embodiments, the pharmaceutically acceptable carrier comprises water, a buffer, or a saline solution. In some embodiments, the composition comprises a liposome. In some embodiments, the pharmaceutically acceptable carrier comprises liposomes, lipids, nanoparticles, proteins, protein-antibody complexes, peptides, cellulose, nanogel, or a combination thereof.

### C. DISEASE TREATMENT AND PREVENTION

The compositions and methods described herein include methods of treating, preventing, or delaying onset of neurodegenerative diseases, such as Parkinson's Disease, Huntington's Disease, and amyotrophic lateral sclerosis (ALS). Also described herein are methods and compositions for including cognition and learning, for use in treating disorders than affect cognition, including Parkinson's Disease, Huntington's Disease, and learning disorders. The compositions and methods reduce neuroinflammation, increase nerve protection, reverse locomotor defects, and activate pathways associated with neuroregeneration.

Disclosed herein, in some embodiments, are methods of treating, preventing or delaying onset of Parkinson's disease, Huntington's disease, ALS, or a learning disorder in a subject in need thereof. Some embodiments include treating Parkinson's disease, Huntington's disease, ALS, or a learning disorder. Some embodiments include preventing Parkinson's disease, Huntington's disease, ALS, or a learning disorder. Some embodiments include delaying onset of Parkinson's disease, Huntington's disease, ALS, or a learning disorder. Some embodiments include treating or delaying onset of Parkinson's disease, Huntington's disease, ALS, or a learning disorder. Some embodiments include administering to the subject a composition described herein. For example, the composition may include ETP69 or a pharmaceutically acceptable salt thereof.

Disclosed herein, in some embodiments, are methods of improving cognition in a subject. In some embodiments, the subject is risk of developing Parkinson's disease, Huntington's disease, ALS, or a learning disorder. Some embodiments include improving cognition in a subject at risk of developing Parkinson's disease, Huntington's disease, ALS, or a learning disorder. Some embodiments include administering to the subject a composition described herein. For example, the composition may include ETP69 or a pharmaceutically acceptable salt thereof.

### 1. Parkinson's Disease

Parkinson's disease is a neurodegenerative disease affecting the motor systems with motor symptoms and cognitive symptoms. Motor symptoms include unintended or uncontrollable movements, such as shaking, stiffness, difficulty walking, difficult talking, and difficulty with balance and coordination. Cognitive symptom include mental and behavioral changes, sleep problems, depression, memory difficulties, and fatigue.

Disclosed herein, in some embodiments, are methods of treating, preventing or delaying onset of Parkinson's disease in a subject in need thereof. Some embodiments include treating Parkinson's disease. Some embodiments include preventing Parkinson's disease. Some embodiments include delaying onset of Parkinson's disease. Some embodiments include treating or delaying onset of Parkinson's disease. Some embodiments include administering to the subject a composition described herein. For example, the composition may include ETP69 or a pharmaceutically acceptable salt thereof.

Disclosed herein, in some embodiments, are methods of improving cognition in a subject. In some embodiments, the subject is risk of developing Parkinson's disease. Some embodiments include improving cognition in a subject at risk of developing Parkinson's disease. Some embodiments include administering to the subject a composition described herein. For example, the composition may include ETP69 or a pharmaceutically acceptable salt thereof.

### 2. Huntington's Disease

Huntington's disease is a neurodegenerative disease resulting from defects in the huntingtin gene. Mutant forms of huntingtin result in an increased decay rate of neurons, including neurons in the basal ganglia. Symptoms include lack of coordination, changes in cognition, motor abnormalities including chorea (hyperkinetic movement disorder), and dementia. Cognition defects include defects in executive function, cognitive flexibility, abstract thinking, rule aquation, and memory defects.

Disclosed herein, in some embodiments, are methods of treating, preventing or delaying onset of Huntington's disease in a subject in need thereof. Some embodiments include treating Huntington's disease. Some embodiments include preventing Huntington's disease. Some embodiments include delaying onset of Huntington's disease. Some embodiments include treating or delaying onset of Huntington's disease. Some embodiments include administering to the subject a composition described herein. For example, the composition may include ETP69 or a pharmaceutically acceptable salt thereof.

Disclosed herein, in some embodiments, are methods of improving cognition in a subject. In some embodiments, the subject is risk of developing Huntington's disease. Some embodiments include improving cognition in a subject at risk of developing Huntington's disease. Some embodiments include administering to the subject a composition described herein. For example, the composition may include ETP69 or a pharmaceutically acceptable salt thereof.

### 3. Amyotrophic Lateral Sclerosis (ALS)

Amyotrophic lateral sclerosis (ALS) is a neurodegenerative disease affecting motor neurons. Symptoms of ALS include stiff muscles, muscle twitches, muscle weakness, muscle wasting, difficulty speaking or swallowing, frontotemporal dementia, and cognitive and behavior dysfunction. Cognitive and behavioral defects include repetition of phrases, loss of inhibition, language dysfunction, executive dysfunction, _{deficits} in social cognition, and deficits in verbal memory.

Disclosed herein, in some embodiments, are methods of treating, preventing or delaying onset of ALS in a subject in need thereof. Some embodiments include treating ALS. Some embodiments include preventing ALS. Some embodiments include delaying onset of ALS. Some embodiments include treating or delaying onset of ALS. Some embodiments include administering to the subject a composition described herein. For example, the composition may include ETP69 or a pharmaceutically acceptable salt thereof.

Disclosed herein, in some embodiments, are methods of improving cognition in a subject. In some embodiments, the subject is risk of developing ALS. Some embodiments include improving cognition in a subject at risk of developing ALS. Some embodiments include administering to the subject a composition described herein. For example, the composition may include ETP69 or a pharmaceutically acceptable salt thereof.

### 4. Learning Disorders

Learning disorders affect the ability of the brain to send, receive and process information. A learning disorder may be present at birth or due to injury developed after birth, including head injuries. Learning disorders include, without limitation, dyslexia, dyscalculia, dysgraphia, auditory processing disorder, language processing disorder, nonverbal learning disabilities, and visual perceptual/visual motor deficits.

Disclosed herein, in some embodiments, are methods of treating, preventing or delaying onset of a learning disorder in a subject in need thereof. Some embodiments include treating a learning disorder. Some embodiments include preventing a learning disorder. Some embodiments include delaying onset of a learning disorder. Some embodiments include treating or delaying onset of a learning disorder. Some embodiments include administering to the subject a composition described herein. For example, the composition may include ETP69 or a pharmaceutically acceptable salt thereof.

Disclosed herein, in some embodiments, are methods of improving cognition in a subject. In some embodiments, the subject is risk of developing a learning disorder. Some embodiments include improving cognition in a subject at risk of developing a learning disorder. Some embodiments include administering to the subject a composition described herein. For example, the composition may include ETP69 or a pharmaceutically acceptable salt thereof.

### D. ADMINISTRATION

In some embodiments, administering the compound (e.g. ETP69) to the subject comprises administering an effective amount of the compound sufficient to inhibit a methyltransferase in the subject. In some embodiments, administering the compound comprises administering an effective amount of the compound sufficient to inhibit SUV39H1. In some embodiments, administering the compound comprises administering an effective amount of the compound sufficient to inhibit ASH1L. In some embodiments, administering the compound comprises administering an effective amount of the compound sufficient to inhibit MLL1 complex. In some embodiments, administering the compound comprises administering an effective amount of the compound sufficient to inhibit MLL4 complex. In some embodiments, administering the compound comprises administering an effective amount of the compound sufficient to inhibit NSD3. In some embodiments, administering the compound comprises administering an effective amount of the compound sufficient to inhibit SET1b complex. In some embodiments, administering the compound comprises administering an effective amount of the compound sufficient to inhibit SMYD2. In some embodiments, administering the compound comprises administering an effective amount of the compound sufficient to inhibit SMYD3. In some embodiments, administering the compound comprises administering an effective amount of the compound sufficient to inhibit SUV39H2. In some embodiments, administering the compound comprises administering an effective amount of the compound sufficient to inhibit a methyltransferase selected from the group consisting of ASH1L, MLL1 complex, MLL4 complex, NSD3, Set1b complex, SMYD2, SMYD3, SUV39H1, and SUV39H2. In some embodiments, administering the compound comprises administering an effective amount of the compound sufficient to inhibit a methyltransferase selected from the group consisting of ASH1L, MLL1 complex, MLL4 complex, NSD3, Set1b complex, SMYD2, SMYD3, SUV39H1, SUV39H2, G9a, MLL2 Complex, MLL3 Complex, NSD1, NSD2, NSD2 (E1099K), NSD2 (T1150A), PRDM9, PRMT7, SETDB1, and SETD2.

In some embodiments, administering the compound inhibits the methyltransferase by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95%. In some embodiments, administering the compound inhibits the methyltransferase by greater than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95%. In some embodiments, the methyltransferase is selected from the group consisting of ASH1L, MLL1 complex, MLL4 complex, NSD3, Set1b complex, SMYD2, SMYD3, SUV39H2, G9a, MLL2 Complex, MLL3 Complex, NSD1, NSD2, NSD2 (E1099K), NSD2 (T1150A), PRDM9, PRMT7, SETDB1, and SETD2. In some embodiments, SUV39H1 is not inhibited.

In some embodiments, the compound is administered at a dose that inhibits a methyltransferase selected from the group consisting of ASH1L, MLL1 complex, MLL4 complex, NSD3, Set1b complex, SMYD2, SMYD3, SUV39H2, G9a, MLL2 Complex, MLL3 Complex, NSD1, NSD2, NSD2 (E1099K), NSD2 (T1150A), PRDM9, PRMT7, SETDB1, and SETD2. In some embodiments, the compound is administered at a dose that does not inhibit SUV39H1.

In some embodiments, the route of administration is intravenous, oral, subcutaneous, intraperitoneal, ocular, intraocular, intramuscular, interstitial, or intracranial. In some embodiments, the administration is systemic. In some embodiments, the administration is intravenous. In some embodiments, the administration is oral. In some embodiments, the administration comprises an injection. In some embodiments, the administration is subcutaneous. In some embodiments, the administration is intraperitoneal. In some embodiments, the administration is ocular. In some embodiments, the administration is intraocular. In some embodiments, the administration is intramuscular. In some embodiments, the administration is interstitial. In some embodiments, the administration is intracranial.

In some embodiments, administering the compound comprises administering a single dose. In some embodiments, administering the compound comprises administering multiple doses (for example, 2 doses). For example, the administration may include multiple doses at separate times. The multiple doses may include 1, 2, 3, 4, 5, 6, 7, 8, 9, 10, 11, 12, 13, 14, 15, 16, 17, 18, 19, or 20, or more doses, or a range of doses defined by any two of the aforementioned numbers of doses. In some embodiments, the administration includes administering 11 doses.

### E. SUBJECTS

Some embodiments of the methods described herein include administration of a compound to a subject. Non-limiting examples of subjects include vertebrates, animals, mammals, dogs, cats, cattle, rodents, mice, rats, primates, monkeys, and humans. In some embodiments, the subject is a vertebrate. In some embodiments, the subject is an animal. In some embodiments, the subject is a mammal. In some embodiments, the subject is a dog. In some embodiments, the subject is a cat. In some embodiments, the subject is a cattle. In some embodiments, the subject is a mouse. In some embodiments, the subject is a rat. In some embodiments, the subject is a primate. In some embodiments, the subject is a monkey. In some embodiments, the subject is an animal, a mammal, a dog, a cat, cattle, a rodent, a mouse, a rat, a primate, or a monkey. In some embodiments, the subject is a human. In some embodiments, the subject is male. In some embodiments, the subject is female.

In some embodiments, the subject is ≥90 years of age. In some embodiments, the subject is ≥85 years of age. In some embodiments, the subject is ≥80 years of age. In some embodiments, the subject is ≥70 years of age. In some embodiments, the subject is ≥60 years of age. In some embodiments, the subject is ≥50 years of age. In some embodiments, the subject is ≥40 years of age. In some embodiments, the subject is ≥30 years of age. In some embodiments, the subject is ≥20 years of age. In some embodiments, the subject is ≥10 years of age. In some embodiments, the subject is ≥1 years of age. In some embodiments, the subject is ≥0 years of age. In some embodiments, the subject is asymptomatic of Parkinson's disease, Huntington's disease, amyotrophic lateral sclerosis (ALS), or a learning disorder. In some embodiments, the subject is at least about 25, about 30, about 35, about 40, about 45, about 50, about, about 55, about 60, about 65, or about 70 years of age.

In some embodiments, the subject is ≤100 years of age. In some embodiments, the subject is ≤90 years of age. In some embodiments, the subject is ≤85 years of age. In some embodiments, the subject is ≤80 years of age. In some embodiments, the subject is ≤70 years of age. In some embodiments, the subject is ≤60 years of age. In some embodiments, the subject is ≤50 years of age. In some embodiments, the subject is ≤40 years of age. In some embodiments, the subject is ≤30 years of age. In some embodiments, the subject is ≤20 years of age. In some embodiments, the subject is ≤10 years of age. In some embodiments, the subject is ≤1 years of age. In some embodiments, the subject is less than about 25, about 30, about 35, about 40, about 45, about 50, about, about 55, about 60, about 65, or about 70 years of age.

In some embodiments, the subject is between 0 and 100 years of age. In some embodiments, the subject is between 20 and 90 years of age. In some embodiments, the subject is between 30 and 80 years of age. In some embodiments, the subject is between 40 and 75 years of age. In some embodiments, the subject is between 50 and 70 years of age. In some embodiments, the subject is between 40 and 85 years of age.

In some embodiments, the subject has Parkinson's disease, Huntington's disease, ALS, or a learning disorder. In some embodiments, the subject is at risk of developing Parkinson's disease, Huntington's disease, ALS, or a learning disorder. In some embodiments, the subject is symptomatic for Parkinson's disease, Huntington's disease, ALS, or a learning disorder. In some embodiments, the subject is asymptomatic of Parkinson's disease, Huntington's disease, ALS, or a learning disorder.

### F. PATIENT SELECTION

In some embodiments, the methods comprise selecting a subject for treatment, wherein a methyltransferase is dysregulated in a tissue of the subject. In some embodiments, the methyltranferase is ASH1L, MLL1 complex, MLL4 complex, NSD3, Set1b complex, SMYD2, SMYD3, SUV39H2, G9a, MLL2 Complex, MLL3 Complex, NSD1, NSD2, NSD2 (E1099K), NSD2 (T1150A), PRDM9, PRMT7, SETDB1, and SETD2. In some embodiments, the methyltransferase is not SUV39H1.

In some embodiments, the methods comprise inhibiting a methyltransferase in a patient in need thereof. In some embodiments, the methyltransferase is ASH1L, MLL1 complex, MLL4 complex, NSD3, Set1b complex, SMYD2, SMYD3, SUV39H2, G9a, MLL2 Complex, MLL3 Complex, NSD1, NSD2, NSD2 (E1099K), NSD2 (T1150A), PRDM9, PRMT7, SETDB1, and SETD2. In some embodiments, the methyltransferase is not SUV39H1.

In some embodiments, the methods comprise selecting a subject for inhibition of a methyltransferase. In some embodiments, the methyltransferase is ASH1L, MLL1 complex, MLL4 complex, NSD3, Set1b complex, SMYD2, SMYD3, SUV39H2, G9a, MLL2 Complex, MLL3 Complex, NSD1, NSD2, NSD2 (E1099K), NSD2 (T1150A), PRDM9, PRMT7, SETDB1, and SETD2. In some embodiments, the methyltransferase is not SUV39H1.

In some embodiments, the subject has, is diagnosed with, or is suspecting of having a cognitive dysfunction. In some embodiments, the cognitive dysfunction is Parkinson's disease, Huntington's disease, ALS, or a learning disorder.

### G. BASELINE CHARACTERISTICS

Some embodiments of the methods described herein include obtaining a baseline measurement from a subject. For example, in some embodiments, a baseline measurement is obtained from the subject prior to treating the subject. In some embodiments, the baseline measurement is a symptom of the Parkinson's disease, Huntington's disease, amyotrophic lateral sclerosis (ALS), or learning disorder such as a symptom described herein. Non-limiting examples of baseline measurements include a baseline memory measurement, a baseline learning measurement, a baseline spontaneous activity measurement, a baseline neuronal architecture measurement, a baseline neuroinflammation measurement, a baseline locomotor measurement, or a baseline biomarker measurement.

In some embodiments, the baseline measurement includes a mental status test. In some embodiments, the mental status test is a Mini-Mental State Exam (MMSE), a Mini-Cog test, a Cantab Mobile test, a Cognigram test, a Cognivue test, or a Cognision and Automated Neuropsychological Assessment Metrics (ANAM) test. In some embodiments, the mental status test is a Mini-Mental State Exam (MMSE). In some embodiments, the mental status test is a Mini-Cog test. In some embodiments, the mental status test is a Cantab Mobile test. In some embodiments, the mental status test is a Cognigram test. In some embodiments, the mental status test is a Cognivue test. In some embodiments, the mental status test is a Cognision and Automated Neuropsychological Assessment Metrics (ANAM) test. In some embodiments, the baseline measurement includes a neuroimaging test. In some embodiments, the neuroimaging test is a magnetic resonance imaging (MRI) or computed tomography (CT). In some embodiments, the neuroimaging test is an MRI test. In some embodiments, the neuroimaging test is a CT test.

In some embodiments, the baseline measurement is obtained directly from the subject. In some embodiments, the baseline measurement is obtained by observation, for example by observation of the subject or of the subject's tissue. In some embodiments, the baseline measurement is obtained noninvasively using an imaging device. In some embodiments, the baseline measurement is obtained in a sample from the subject. In some embodiments, the baseline measurement is obtained in one or more histological tissue sections. In some embodiments, the baseline measurement is obtained by performing an assay such as an immunoassay, a colorimetric assay, or a fluorescence assay, on the sample obtained from the subject. In some embodiments, the baseline measurement is obtained by an immunoassay, a colorimetric assay, or a fluorescence assay. In some embodiments, the baseline measurement is obtained by PCR.

In some embodiments, the baseline measurement is a baseline memory measurement. In some embodiments, the baseline memory measurement is a baseline spatial memory measurement. In some embodiments, the baseline memory measurement is a baseline hippocampal-based spatial memory measurement. In some embodiments, the baseline spatial memory measurement comprises a fear-based test. In some embodiments, the baseline spatial memory measurement comprises a fear-conditioning test.

In some embodiments, the baseline measurement is a baseline learning measurement. In some embodiments, the baseline learning measurement is a baseline visual-cognitive memory and learning measurement. In some embodiments, the baseline visual-cognitive memory and learning measurement includes a color memory and learning measurement. In some embodiments, the baseline visual-cognitive memory and learning measurement includes a contrast memory and learning measurement. In some embodiments, the baseline visual-cognitive memory and learning measurement includes a transition memory and learning measurement. In some embodiments, the baseline visual-cognitive memory and learning measurement includes a spatial memory and learning measurement. In some embodiments, the baseline measurement is a baseline spontaneous activity measurement.

In some embodiments, the baseline measurement is a baseline neuronal architecture measurement. In some embodiments, the baseline neuronal architecture measurement includes a baseline spine integrity measurement. In some embodiments, the baseline neuronal architecture measurement includes a baseline dendritic spine measurement. In some embodiments, the baseline dendritic spine measurement assesses long-thin filopodia, long thin, thin, stubby, wide-headed mushroom, and/or branched spines. In some embodiments, the baseline neuronal architecture measurement includes a baseline spine density measurement. In some embodiments, the baseline neuronal architecture measurement includes a number of synapses. In some embodiments, the baseline neuronal architecture measurement is determined in a biopsy. In some embodiments, the baseline neuronal architecture measurement is determined using a stain such as a Golgi-Cox stain. In some embodiments, the baseline neuronal architecture measurement is determined using photography. In some embodiments, the baseline neuronal architecture measurement is determined using microscopy.

In some embodiments, the baseline measurement includes a baseline neuroinflammation measurement. In some embodiments, the baseline neuroinflammation measurement includes a baseline activated or a baseline reactive immune activation measurement. In some embodiments, the baseline neuroinflammation measurement includes a baseline activated or a baseline reactive immune cell measurement. In some embodiments, the baseline neuroinflammation measurement includes a baseline reactive astrocyte measurement. In some embodiments, the baseline neuroinflammation measurement includes a baseline activated microglia measurement. In some embodiments, the baseline neuroinflammation measurement includes a baseline macrophage measurement. In some embodiments, the baseline neuroinflammation measurement is obtained in a tissue or fluid sample. In some embodiments, the baseline neuroinflammation measurement is obtained from a biopsy. In some embodiments, the baseline neuroinflammation measurement is obtained by an assay such as an immunoassay, by fluorescence-activated Cell Sorting (FACS), or by histological assessment.

In some embodiments, the baseline measurement is a baseline locomotor measurement. In some embodiments, the baseline locomotor measurement is a measurement of respiratory muscle function. In some embodiments, the baseline measurement of respiratory muscle function is a forced vital capacity measurement, a maximal inspiratory pressure measurement, or a maximal expiratory pressure measurement. In some embodiments, the baseline locomotor measurement in a functional rate measurement. In some embodiments, the functional rating measurement is the Appel ALS rating scale, the ALS functional rating scale, the Unified Huntington Disease Rating Scale (UHDRS), Total Functional Capacity (TFC) Scale, or a Total Motor Score (TMS).

In some embodiments, the baseline measurement is a baseline molecular marker measurement. In some embodiments, the baseline molecular marker measurement is a baseline histone trimethylation (H3K9me3) measurement. In some embodiments, the baseline molecular measurement is a baseline nucleosome methylation measurement. In some embodiments, the baseline measurement is a baseline core histone methylation measurement. In some embodiments, the baseline measurement is a histone H4 methylation measurement. In some embodiments, the baseline measurement isa MEKK2 methylation measurement. In some embodiments, the baseline molecular measurement is a measurement of an enzyme from Table 1. In some embodiments, the baseline molecular measurement is a measurement of ASH1L, MLL1 complex, MLL4 complex, NSD3, Set1b complex, SMYD2, SMYD3, SUV39H2, G9a, MLL2 Complex, MLL3 Complex, NSD1, NSD2, NSD2 (E1099K), NSD2 (T1150A), PRDM9, PRMT7, SETDB1, and SETD2. In some embodiments, the baseline molecular marker measurement is a baseline protein measurement. In some embodiments, the baseline molecular marker measurement is a baseline biomarker measurement. In some embodiments, the baseline molecular marker measurement is determined in a biopsy. In some embodiments, the baseline molecular marker measurement is determined using an immunoassay such as an ELISA.

Some embodiments of the methods described herein include obtaining a sample from a subject. In some embodiments, the baseline measurement is obtained in a sample obtained from the subject. In some embodiments, the sample is obtained from the subject prior to administration or treatment of the subject with a composition described herein. In some embodiments, a baseline measurement is obtained in a sample obtained from the subject prior to administering the composition to the subject.

In some embodiments, the sample comprises a fluid. In some embodiments, the sample is a fluid sample. In some embodiments, the sample is a blood, plasma, or serum sample. In some embodiments, the sample comprises blood. In some embodiments, the sample is a blood sample. In some embodiments, the sample is a whole-blood sample. In some embodiments, the blood is fractionated or centrifuged. In some embodiments, the sample comprises plasma. In some embodiments, the sample is a plasma sample. In some embodiments, the sample comprises serum. In some embodiments, the sample is a serum sample. In some embodiments, the sample comprises cerebrospinal fluid (CSF).

In some embodiments, the sample comprises a tissue. In some embodiments, the sample is a tissue sample. In some embodiments, the sample comprises neural tissue. In some embodiments, the sample is a brain sample. In some embodiments, the sample is a hippocampal sample. In some embodiments, the sample comprises neurons.

In some embodiments, the sample comprises cells. The cells may include neural cells. The cells may include cerebral cells. The cells may include cerebral macrophages, microglia, or astrocytes. In some embodiments, the cells include neurons. In some embodiments, the cells include macrophages. In some embodiments, the cells include microglia. In some embodiments, the cells include astrocytes.

### H. TREATMENT EFFECTS

In some embodiments, the composition or administration of the composition affects a measurement such as a memory measurement, a learning measurement, a spontaneous activity measurement, a neuronal architecture measurement, a neuroinflammation measurement, or a biomarker measurement, relative to the baseline measurement. In some embodiments, the measurement is related to a symptom of Parkinson's disease, Huntington's disease, amyotrophic lateral sclerosis (ALS), or a learning disorder.

Some embodiments of the methods described herein include obtaining the measurement from a subject. For example, the measurement may be obtained from the subject after treating the subject. In some embodiments, the measurement is obtained in a second sample (such as a fluid or tissue sample described herein) obtained from the subject after the composition is administered to the subject. In some embodiments, the measurement is an indication that the disorder has been treated.

In some embodiments, the measurement is obtained directly from the subject. In some embodiments, the measurement is obtained noninvasively using an imaging device. In some embodiments, the measurement is obtained in a second sample from the subject. In some embodiments, the measurement is obtained in one or more histological tissue sections. In some embodiments, the measurement is obtained by performing an assay on the second sample obtained from the subject. In some embodiments, the measurement is obtained by an assay, such as an assay described herein. In some embodiments, the assay is an immunoassay, a colorimetric assay, a fluorescence assay, or a PCR assay. In some embodiments, the measurement is obtained by an assay such as an immunoassay, a colorimetric assay, or a fluorescence assay. In some embodiments, the measurement is obtained by PCR. In some embodiments, the measurement is obtained by histology. In some embodiments, the measurement is obtained by observation. In some embodiments, additional measurements are made, such as in a 3rd sample, a 4th sample, or a fifth sample.

In some embodiments, the measurement is obtained within 1 hour, within 2 hours, within 3 hours, within 4 hours, within 5 hours, within 6 hours, within 12 hours, within 18 hours, or within 24 hours after the administration of the composition. In some embodiments, the measurement is obtained within 1 day, within 2 days, within 3 days, within 4 days, within 5 days, within 6 days, or within 7 days after the administration of the composition. In some embodiments, the measurement is obtained within 1 week, within 2 weeks, within 3 weeks, within 1 month, within 2 months, within 3 months, within 6 months, within 1 year, within 2 years, within 3 years, within 4 years, or within 5 years after the administration of the composition. In some embodiments, the measurement is obtained after 1 hour, after 2 hours, after 3 hours, after 4 hours, after 5 hours, after 6 hours, after 12 hours, after 18 hours, or after 24 hours after the administration of the composition. In some embodiments, the measurement is obtained after 1 day, after 2 days, after 3 days, after 4 days, after 5 days, after 6 days, or after 7 days after the administration of the composition. In some embodiments, the measurement is obtained after 1 week, after 2 weeks, after 3 weeks, after 1 month, after 2 months, after 3 months, after 6 months, after 1 year, after 2 years, after 3 years, after 4 years, or after 5 years, following the administration of the composition.

In some embodiments, the composition reduces the measurement relative to the baseline measurement. In some embodiments, the reduction is measured in a second tissue sample obtained from the subject after administering the composition to the subject. In some embodiments, the reduction is measured directly in the subject after administering the composition to the subject. In some embodiments, the measurement is decreased by about 2.5% or more, about 5% or more, or about 7.5% or more, relative to the baseline measurement. In some embodiments, the measurement is decreased by about 10% or more, relative to the baseline measurement. In some embodiments, the measurement is decreased by about 20% or more, about 30% or more, about 40% or more, about 50% or more, about 60% or more, about 70% or more, about 80% or more, about 90% or more, relative to the baseline measurement. In some embodiments, the measurement is decreased by no more than about 2.5%, no more than about 5%, or no more than about 7.5%, relative to the baseline measurement. In some embodiments, the measurement is decreased by no more than about 10%, relative to the baseline measurement. In some embodiments, the measurement is decreased by no more than about 20%, no more than about 30%, no more than about 40%, no more than about 50%, no more than about 60%, no more than about 70%, no more than about 80%, no more than about 90%, or no more than about 100% relative to the baseline measurement. In some embodiments, the measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the composition increases the measurement relative to the baseline measurement. In some embodiments, the increase is measured in a second tissue sample obtained from the subject after administering the composition to the subject. In some embodiments, the increase is measured directly in the subject after administering the composition to the subject. In some embodiments, the measurement is increased by about 2.5% or more, about 5% or more, or about 7.5% or more, relative to the baseline measurement. In some embodiments, the measurement is increased by about 10% or more, relative to the baseline measurement. In some embodiments, the measurement is increased by about 20% or more, about 30% or more, about 40% or more, about 50% or more, about 60% or more, about 70% or more, about 80% or more, about 90% or more, relative to the baseline measurement. In some embodiments, the measurement is increased by about 100% or more, increased by about 250% or more, increased by about 500% or more, increased by about 750% or more, or increased by about 1000% or more, relative to the baseline measurement. In some embodiments, the measurement is increased by no more than about 2.5%, no more than about 5%, or no more than about 7.5%, relative to the baseline measurement. In some embodiments, the measurement is increased by no more than about 10%, relative to the baseline measurement. In some embodiments, the measurement is increased by no more than about 20%, no more than about 30%, no more than about 40%, no more than about 50%, no more than about 60%, no more than about 70%, no more than about 80%, no more than about 90%, or no more than about 100% relative to the baseline measurement. In some embodiments, the measurement is increased by no more than about 100%, increased by no more than about 250%, increased by no more than about 500%, increased by no more than about 750%, or increased by no more than about 1000%, relative to the baseline measurement. In some embodiments, the measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement includes a mental status test. In some embodiments, the mental status test is a Mini-Mental State Exam (MMSE), a Mini-Cog test, a Cantab Mobile test, a Cognigram test, a Cognivue test, or a Cognision and Automated Neuropsychological Assessment Metrics (ANAM) test. In some embodiments, the measurement includes a neuroimaging test. In some embodiments, the neuroimaging test is a magnetic resonance imaging (MRI) or computed tomography (CT).

In some embodiments, the measurement is a memory measurement. In some embodiments, the memory measurement is a spatial memory measurement. In some embodiments, the memory measurement is a hippocampal-based spatial memory measurement. In some embodiments, the spatial memory measurement comprises a fear-based test. In some embodiments, the spatial memory measurement comprises a fear-conditioning test. In some embodiments, the memory measurement is improved. In some embodiments, the memory measurement is increased.

In some embodiments, the measurement is a learning measurement. In some embodiments, the learning measurement is a visual-cognitive memory and learning measurement. In some embodiments, the visual-cognitive memory and learning measurement includes a color memory and learning measurement. In some embodiments, the visual-cognitive memory and learning measurement includes a contrast memory and learning measurement. In some embodiments, the visual-cognitive memory and learning measurement includes a transition memory and learning measurement. In some embodiments, the visual-cognitive memory and learning measurement includes a spatial memory and learning measurement. In some embodiments, the learning measurement is improved. In some embodiments, the learning measurement is increased. In some embodiments, the measurement is a spontaneous activity measurement. In some embodiments, the spontaneous activity measurement is improved. In some embodiments, the spontaneous activity measurement is increased.

In some embodiments, the measurement is a neuronal architecture measurement. In some embodiments, the neuronal architecture measurement includes a spine integrity measurement. In some embodiments, the neuronal architecture measurement includes a dendritic spine measurement. In some embodiments, the dendritic spine measurement assesses long-thin filopodia, long thin, thin, stubby, wide-headed mushroom, and/or branched spines. In some embodiments, the neuronal architecture measurement includes a spine density measurement. In some embodiments, the neuronal architecture measurement includes a number of synapses. In some embodiments, the neuronal architecture measurement is determined in a biopsy. In some embodiments, the neuronal architecture measurement is determined using a stain such as a Golgi-Cox stain. In some embodiments, the neuronal architecture measurement is determined using photography. In some embodiments, the neuronal architecture measurement is determined using microscopy. In some embodiments, the neuronal architecture measurement is improved. In some embodiments, the neuronal architecture measurement (e.g. number of synapses) is increased.

In some embodiments, the measurement includes a neuroinflammation measurement. In some embodiments, the neuroinflammation measurement includes an activated or a reactive immune activation measurement. In some embodiments, the neuroinflammation measurement includes an activated or a reactive immune cell measurement. In some embodiments, the neuroinflammation measurement includes a reactive astrocyte measurement. In some embodiments, the neuroinflammation measurement includes an activated microglia measurement. In some embodiments, the neuroinflammation measurement includes a macrophage measurement. In some embodiments, the neuroinflammation measurement is obtained in a tissue or fluid sample. In some embodiments, the neuroinflammation measurement is obtained from a biopsy. In some embodiments, the neuroinflammation measurement is obtained by an assay such as an immunoassay, by fluorescence-activated Cell Sorting (FACS), or by histological assessment.

In some embodiments, the measurement is a locomotor measurement. In some embodiments, the locomotor measurement is a measurement of respiratory muscle function. In some embodiments, the measurement of respiratory muscle function is a forced vital capacity measurement, a maximal inspiratory pressure measurement, or a maximal expiratory pressure measurement. In some embodiments, the locomotor measurement in a functional rate measurement. In some embodiments, the functional rating measurement is the Appel ALS rating scale, the ALS functional rating scale, the Unified Huntington Disease Rating Scale (UHDRS), Total Functional Capacity (TFC) Scale, or a Total Motor Score (TMS). In some embodiments, the locomotor measurement is improved relative to the baseline locomotor measurement.

In some embodiments, the measurement is a molecular marker measurement. In some embodiments, the molecular marker measurement is a histone trimethylation (H3K9me3) measurement. In some embodiments, the molecular marker measurement is a nucleosome methylation measurement. In some embodiments, the molecular marker measurement is a core histone methylation measurement. In some embodiments, the molecular marker measurement is a histone H4 methylation measurement. In some embodiments, the molecular marker measurement isa MEKK2 methylation measurement. In some embodiments, the molecular measurement is a measurement of an enzyme from Table 1. In some embodiments, the molecular measurement is a measurement of ASH1L, MLL1 complex, MLL4 complex, NSD3, Set1b complex, SMYD2, SMYD3, SUV39H2, G9a, MLL2 Complex, MLL3 Complex, NSD1, NSD2, NSD2 (E1099K), NSD2 (T1150A), PRDM9, PRMT7, SETDB1, and SETD2. In some embodiments, the molecular marker measurement is a protein measurement. In some embodiments, the molecular marker measurement is a biomarker measurement. The measurement may be increased, relative to the baseline measurement. The measurement may be decreased, relative to the baseline measurement. In some embodiments, the molecular marker measurement is determined in a biopsy. In some embodiments, the molecular marker measurement is determined using an immunoassay such as an ELISA. In some embodiments, the molecular marker measurement is increased. For example, the BDNF measurement may be increased following treatment with the compound. In some embodiments, the molecular marker measurement is decreased. For example, the H3K9me3 measurement may be decreased following treatment with the compound.

In some embodiments, the administration improves a symptom of the Parkinson's disease, Huntington's disease, ALS, or a learning disorder. In some embodiments, the administration reduces a symptom of the Parkinson's disease, Huntington's disease, ALS, or a learning disorder. In some embodiments, the administration prevents a symptom of the Parkinson's disease, Huntington's disease, ALS, or a learning disorder. In some embodiments, the administration delays a symptom of the Parkinson's disease, Huntington's disease, ALS, or a learning disorder. In some embodiments, the administration slows progression of a symptom of the Parkinson's disease, Huntington's disease, ALS, or a learning disorder.

Described herein, are methods of delaying onset of Parkinson's disease, Huntington's disease, ALS, or a learning disorder. In some embodiments, the delaying onset of Parkinson's disease, Huntington's disease, ALS, or a learning disorder comprises a delay in onset of at least one symptom of Parkinson's disease, Huntington's disease, ALS, or a learning disorder. In some embodiments, the delay in onset of at least on symptom is at least about 6 months, about 12 months, about 18 months, about 2 years, about 3 years, about 5 years, about 10 years, about 15 years, or about 20 years. In some embodiments, the delay in onset of at least on symptom is at least 6 months. In some embodiments, the delay in onset of at least on symptom is at least 12 months. In some embodiments, the delay in onset of at least on symptom is at least 18 months. In some embodiments, the delay in onset of at least on symptom is at least 2 years. In some embodiments, the delay in onset of at least on symptom is at least 3 years. In some embodiments, the delay in onset of at least on symptom is at least 5 years. In some embodiments, the delay in onset of at least on symptom is at least 10 years. In some embodiments, the delay in onset of at least on symptom is at least 15 years. In some embodiments, the delay in onset of at least on symptom is at least 20 years.

In some embodiments, the delaying onset of Parkinson's disease, Huntington's disease, ALS, or a learning disorder comprises a delay in onset of no more than one symptom of Parkinson's disease, Huntington's disease, ALS, or a learning disorder. In some embodiments, the delay in onset of no more than on symptom is no more than about 6 months, about 12 months, about 18 months, about 2 years, about 3 years, about 5 years, about 10 years, about 15 years, or about 20 years. In some embodiments, the delay in onset of no more than on symptom is no more than 6 months. In some embodiments, the delay in onset of no more than on symptom is no more than 12 months. In some embodiments, the delay in onset of no more than on symptom is no more than 18 months. In some embodiments, the delay in onset of no more than on symptom is no more than 2 years. In some embodiments, the delay in onset of no more than on symptom is no more than 3 years. In some embodiments, the delay in onset of no more than on symptom is no more than 5 years. In some embodiments, the delay in onset of no more than on symptom is no more than 10 years. In some embodiments, the delay in onset of no more than on symptom is no more than 15 years. In some embodiments, the delay in onset of no more than on symptom is no more than 20 years

Described herein, are methods of delaying onset of at least one symptom of Parkinson's disease, Huntington's disease, ALS, or a learning disorder. In some embodiments, the symptom comprises memory loss, difficulty concentrating, difficulty completing familiar tasks, confusion with time or place, difficulty understanding visual images and spatial relationships, language difficulties, misplacing items, decreased or poor judgement, social withdrawal, and/or mood or personality changes. In some embodiments, the symptom includes memory loss. In some embodiments, the symptom includes difficulty concentrating. In some embodiments, the symptom includes difficulty completing familiar tasks. In some embodiments, the symptom includes confusion with time or place. In some embodiments, the symptom includes difficulty understanding visual images and spatial relationships. In some embodiments, the symptom includes language difficulties. In some embodiments, the symptom includes misplacing items. In some embodiments, the symptom includes decreased or poor judgement. In some embodiments, the symptom includes social withdrawal. In some embodiments, the symptom includes mood or personality changes.

In some embodiments, the method improves memory loss, difficulty concentrating, difficulty completing familiar tasks, confusion with time or place, difficulty understanding visual images and spatial relationships, language difficulties, misplacing items, decreased or poor judgement, social withdrawal, and/or mood or personality changes in the subject. In some embodiments, the method improves memory loss. In some embodiments, the method improves difficulty concentrating. In some embodiments, the method improves difficulty completing familiar tasks. In some embodiments, the method improves confusion with time or place. In some embodiments, the method improves difficulty understanding visual images. In some embodiments, the method improves difficulty understanding spatial relationships. In some embodiments, the method improves difficulty understanding visual images and spatial relationships. In some embodiments, the method improves language difficulties. In some embodiments, the method improves misplacing items. In some embodiments, the method improves decreased or poor judgement. In some embodiments, the method improves social withdrawal. In some embodiments, the method improves mood or personality changes.

In some embodiments, the treatment results in improvement in a mental status test and/or a neuroimaging test. In some embodiments, the treatment results in improvement in a mental status test. In some embodiments, the mental status test is a Mini-Mental State Exam (MMSE), a Mini-Cog test, a Cantab Mobile test, a Cognigram test, a Cognivue test, or a Cognision and Automated Neuropsychological Assessment Metrics (ANAM) test. In some embodiments, the mental status test is a Mini-Mental State Exam (MMSE). In some embodiments, the mental status test is a Mini-Cog test. In some embodiments, the mental status test is a Cantab Mobile test. In some embodiments, the mental status test is a Cognigram test. In some embodiments, the mental status test is a Cognivue test. In some embodiments, the mental status test is a Cognision and Automated Neuropsychological Assessment Metrics (ANAM) test. In some embodiments, the improvement comprises an improved score relative to a score obtained prior to administration of the composition.

In some embodiments, the treatment results in improvement in a neuroimaging test. In some embodiments, the neuroimaging test is a magnetic resonance imaging (MRI) or computed tomography (CT). In some embodiments, the neuroimaging test is an MRI test. In some embodiments, the neuroimaging test is a CT test.

### I. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates. Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like.

The terms “subject,” and “patient” may be used interchangeably herein. A “subject” can be a biological entity containing expressed genetic materials. The biological entity can be a plant, animal, or microorganism, including, for example, bacteria, viruses, fungi, and protozoa. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### J. EMBODIMENTS

In certain aspects, disclosed herein are the following embodiments:
1. A method of treating Parkinson's disease, Huntington's disease, ALS, or a learning disorder in a subject in need thereof, the method comprising administering to the subject a compound comprising an inhibitor of trimethylation of histone 3, lysine 9 (H3K9).
2. The method of embodiment 1, wherein the compound comprises an inhibitor of SUV39H1.
3. The method of embodiment 1, wherein the compound comprises an inhibitor of SUV39H2.
4. The method of any one of embodiments 1-3, wherein the compound has the structure of Formula (I), or a pharmaceutically acceptable salt thereof:

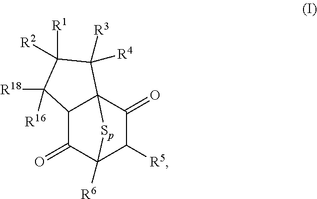

wherein, p is 2, 3 or 4, and
R¹, R², R³, R⁴, R⁵, R⁶, R¹⁶, and R¹⁸ are independently hydrogen, halogen, —N₃, —CF₃, —CCl₃, —CBr₃, —Cl₃, —CN, —CHO, —OH, —NH₂, —COOH, —CONH₂, —NO₂, —SH, —SO₂, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.
5. The method of any one of embodiments 1-4, wherein the compound comprises (Rac-(3S,6S,7S,8aS)-6-(benzo[d][1,3]dioxol-5-yl)-2,3,7-trimethyl-1,4-dioxohexahydro-6H-3,8a-epidithiopyrrolo[1,2-a]pyrazine-7-carbonitrile) (ETP69), or a pharmaceutically acceptable salt thereof.
6. The method of any one of embodiments 1-5, wherein the administration improves locomotor activity in the subject.
7. The method of any one of embodiments 1-6, wherein the administration reduces neuroinflammation in the subject.
8. The method of any one of embodiments 1-7, wherein the administration increases or improves a neuroprotective phenotype.
9. The method of embodiment 7, wherein the administration activates brain-derived neurotrophic factor (BDNF) or VGF nerve growth factor inducible (VGF) signaling in the subject.
10. The method of any one of embodiments 1-9, wherein the administration reduces or improves a phenotype associated with Parkinson's disease, Huntington's disease, ALS, or the learning disorder in the subject.
11. The method of embodiment 10, wherein the subject has or has been diagnosed with Parkinson's disease.
12. The method of embodiment 10, wherein the subject has or has been diagnosed with Huntington's disease.
13. The method of embodiment 10, wherein the subject has or has been diagnosed with ALS.
14. The method of embodiment 10, wherein the subject has or has been diagnosed with the learning disorder.
15. A method of inhibiting an enzyme selected from the group consisting of: ASH1L, MLL1 complex, MLL4 complex, NSD3, Set1b complex, SMYD2, SMYD3, SUV39H1, and SUV39H2, comprising contacting the enzyme with a compound having the structure of Formula (I), or a pharmaceutically acceptable salt thereof:

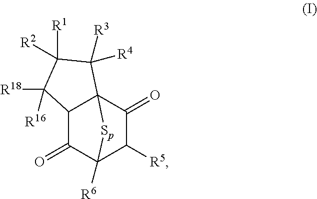

wherein, p is 2, 3 or 4, and
R¹, R², R³, R⁴, R⁵, R⁶, R¹⁶, and R¹⁸ are independently hydrogen, halogen, —N₃, —CF₃, —CCl₃, —CBr₃, —Cl₃, —CN, —CHO, —OH, —NH₂, —COOH, —CONH₂, —NO₂, —SH, —SO₂, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.
16. The method of embodiment 15, wherein the compound comprises (Rac-(3S,6S,7S,8aS)-6-(benzo[d][1,3]dioxol-5-yl)-2,3,7-trimethyl-1,4-dioxohexahydro-6H-3,8a-epidithiopyrrolo[1,2-a]pyrazine-7-carbonitrile) (ETP69), or a pharmaceutically acceptable salt thereof.
17. The method of embodiment 15 or 16, wherein the contact is in vitro.
18. The method of embodiment 15 or 16, wherein the contact is in vivo.
19. The method of embodiment 18, wherein the contact is in a neuron of a subject.
20. The method of embodiment 18 or 19, wherein the contact is in a human subject.
21. The method of any one of embodiments 15-21, further comprising assessing an activity of the enzyme during or after the contact of the enzyme with the compound.
22. The method of embodiment 21, wherein the activity of the enzyme is decreased by at least 75% after the contact of the enzyme with the compound.

### EXAMPLES

The following examples are included for illustrative purposes only and are not intended to limit the scope of the invention.

### Example 1: ETP69 Inhibition of Methyltransferases

The ability of ETP69 to inhibit methyltransferases was tested. Results are depicted in Table 1. ASH1L, MLL1 complex, MLL4 complex, NSD3, Set1b complex, SMYD2, SMYD3, SUV39H1, SUV39H2, G9a, MLL2 Complex, MLL3 Complex, NSD1, NSD2, NSD2 (E1099K), NSD2 (T1150A), PRDM9, PRMT7, SETDB1, and SETD2 all showed inhibition of at least 7500 or more relative to DMSO controls.

TABLE 1
ETP69 inhibition of methyltransferases

| | | | | Enzyme % Activity | | | |
|---|---|---|---|---|---|---|---|
| | | | | (relative to DMSO | | | |
| | | | | controls) | | | |
| | | | | Compound ID: | | | |
| | | | | NT1721 | | | |
| | | | | Testing Concen- | | | |
| | | | | tration (μM): | | | |
| | | Substrate | | 20 | | Control | |
| Methyltransferase: | Substrate: | Concentration: | | Data 1: | Data 2: | IC50* (M): | Control ID: |
| ASH1L | Nucleosomes | 0.05 | mg/mL | 3.42 | 4.17 | 3.08E−08 | Chaetocin |
| DNMT1 | Poly dl-dC | 0.001 | mg/mL | 104.89 | 108.11 | 2.07E−07 | SAH |
| DNMT3a | Lambda DNA | 0.0075 | mg/mL | 76.81 | 78.12 | 1.54E−07 | SAH |
| DNMT3b | Lambda DNA | 0.0075 | mg/mL | 84.82 | 80.89 | 3.79E−08 | SAH |
| DNMT3b/3L | Lambda DNA | 0.0075 | mg/mL | 112.74 | 110.62 | 1.93E−08 | SAH |
| DOT1L | Nucleosomes | 0.05 | mg/mL | 103.85 | 98.45 | 2.40E−07 | SAH |
| EZH1 Complex | Core Histone | 0.05 | mg/mL | 103.44 | 104.86 | 2.93E−05 | SAH |
| EZH2 Complex | Core Histone | 0.05 | mg/mL | 98.37 | 97.85 | 3.25E−05 | SAH |
| EZH2(Y641F) | Core Histone | 0.05 | mg/mL | 102.54 | 102.95 | 5.47E−05 | SAH |
| Complex | | | | | | | |
| G9a | Histone H3 1-21 | 2.5 | μM | 40.87 | 38.73 | 1.30E−06 | SAH |
| GLP | Histone H3 1-21 | 2.5 | μM | 91.37 | 94.17 | 5.53E−07 | SAH |
| METTL21A | HSPA8-[CTD] | 2.5 | μM | 78.64 | 78.51 | 6.46E−05 | SAH |
| MLL1 Complex | Nucleosomes | 0.05 | mg/mL | 5.08 | 5.33 | 2.05E−07 | SAH |
| MLL2 Complex | Nucleosomes | 0.05 | mg/mL | 19.04 | 20.42 | 2.12E−05 | SAH |
| MLL3 Complex | Nucleosomes | 0.05 | mg/mL | 36.58 | 32.63 | 1.89E−05 | SAH |
| MLL4 Complex | Nucleosomes | 0.05 | mg/mL | 1.32 | 1.17 | 8.16E−07 | SAH |
| NRMT1 | RCC1 | 5 | μM | 90.50 | 94.62 | 3.10E−07 | SAH |
| NSD1 | Nucleosomes | 0.05 | mg/mL | 34.29 | 36.14 | 7.51E−06 | SAH |
| NSD2 | Nucleosomes | 0.05 | mg/mL | 20.00 | 19.85 | 3.36E−06 | SAH |
| NSD2 (E1099K) | Nucleosomes | 0.05 | mg/mL | 32.33 | 32.74 | 1.58E−06 | SAH |
| NSD2 (T1150A) | Nucleosomes | 0.05 | mg/mL | 23.87 | 25.49 | 4.72E−07 | SAH |
| NSD3 | Nucleosomes | 0.05 | mg/mL | 10.33 | 10.21 | 8.52E−08 | Chaetocin |
| PRDM9 | Histone H3 | 5 | μM | 46.22 | 47.13 | 4.69E−06 | Chaetocin |
| PRMT1 | Histone H4 | 5 | μM | 113.63 | 114.87 | 1.41E−07 | SAH |
| PRMT3 | Histone H4 | 5 | μM | 93.96 | 95.00 | 8.70E−07 | SAH |
| PRMT4 | Histone H3 | 5 | μM | 115.45 | 114.37 | 1.12E−07 | SAH |
| PRMT5/MEP50 | Histone H2A | 5 | μM | 92.22 | 93.10 | 4.91E−07 | SAH |
| Complex | | | | | | | |
| PRMT5 | Histone H2A | 5 | μM | 121.25 | 123.34 | 7.96E−07 | SAH |
| (C449S)/MEP50 | | | | | | | |
| PRMT6 | GST-GAR | 5 | μM | 128.26 | 128.87 | 2.04E−07 | SAH |
| PRMT7 | GST-GAR | 5 | μM | 66.70 | 66.73 | 1.33E−07 | SAH |
| PRMT8 | Histone H4 | 5 | μM | 95.70 | 94.72 | 5.62E−08 | SAH |
| SET1b Complex | Core Histone | 0.05 | mg/mL | 0.60 | 0.47 | 8.59E−06 | SAH |
| SET7/9 | Core Histone | 0.05 | mg/mL | 107.17 | 106.78 | 8.29E−05 | SAH |
| SET8 | Nucleosomes | 0.05 | mg/mL | 92.31 | 92.56 | 1.09E−07 | Ryuvidine |
| SETDB1 | Histone H3 | 5 | μM | 40.06 | 37.13 | 1.04E−06 | SAH |
| SETD2 | Nucleosomes | 0.05 | mg/mL | 20.95 | 22.89 | 3.06E−06 | SAH |
| SMYD2 | Histone H4 | 5 | μM | 4.23 | 4.97 | 9.20E−07 | LLY 507 |
| SMYD3 | MEKK2 | 0.5 | μM | 8.99 | 7.71 | 1.42E−05 | SAH |
| SUV39H1 | Histone H3 | 5 | μM | 2.36 | 2.53 | 8.38E−05 | SAH |
| SUV39H2 | Histone H3 | 5 | μM | 4.40 | 4.40 | 4.43E−05 | SAH |
| SUV420H1TV2 | Nucleosomes | 0.05 | mg/mL | 91.49 | 89.95 | 1.17E−04 | SAH |

### Example 2: Treatment with ETP69 Restored Brain Synaptic Density

The brain of 18-month old mice was stained with the Golgi-Cox method to detect the cytoarchitecture and morphology of neurons. FIG. 1A depicts representative images of the cingulate cortex (cc) of mice administered DMSO or ETP69. FIG. 1B depicts a representative hippocampus. FIG. 1C depicts a representative neuron from a DMSO treated and ETP69 treated hippocampus. FIG. 1D is a schematic of the quantification of neuronal spines. Mice treated with ETP69 showed an increase in both thin spines and dendritic spines, in both the cc and the hippocampus (FIGS. 1E-1H).

### Example 3: Treatment with ETP69 Reduced Epigenome Histone Methylation and Disease-Related Gene Silencing

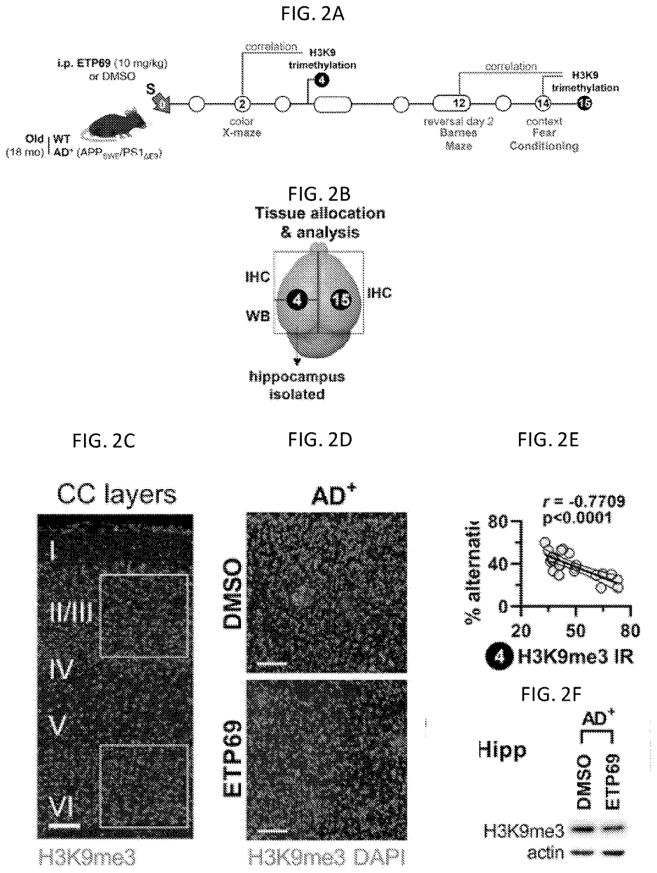

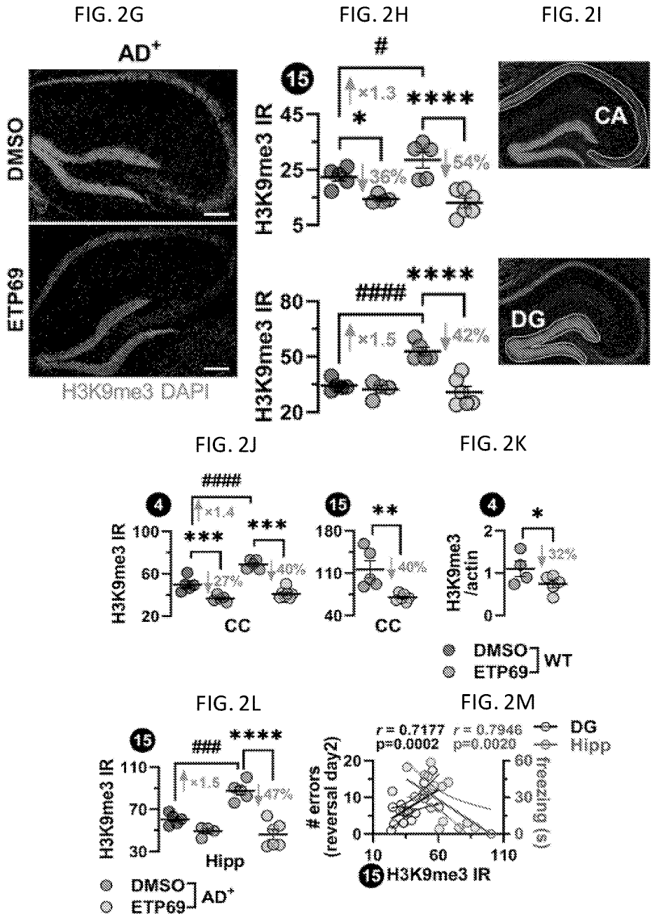

The experimental protocol is depicted in FIG. 2A. Mice were treated with ETP69 or DMSO. After 2 days, they were tested on the color X-maze. At day 12, they were tested on the Barnes maze. On day 14, they were tested for fear conditioning. Brain tissue was isolated as depicted for analysis of H3K9me3 staining on days 4 and 15, as depicted in FIG. 2B-2D. Levels of H3K9me3 correlated with the percent of alterations at day 4 as depicted in FIG. 2E. At day 15, H3K9me3 was decreased in mice treated with ETP69 as compared to mice treated with DMSO. Levels of H3K9me3 was decreased in both the cingulate cortex and the hippocampus at 4 and 15 days (FIGS. 2J-2M).

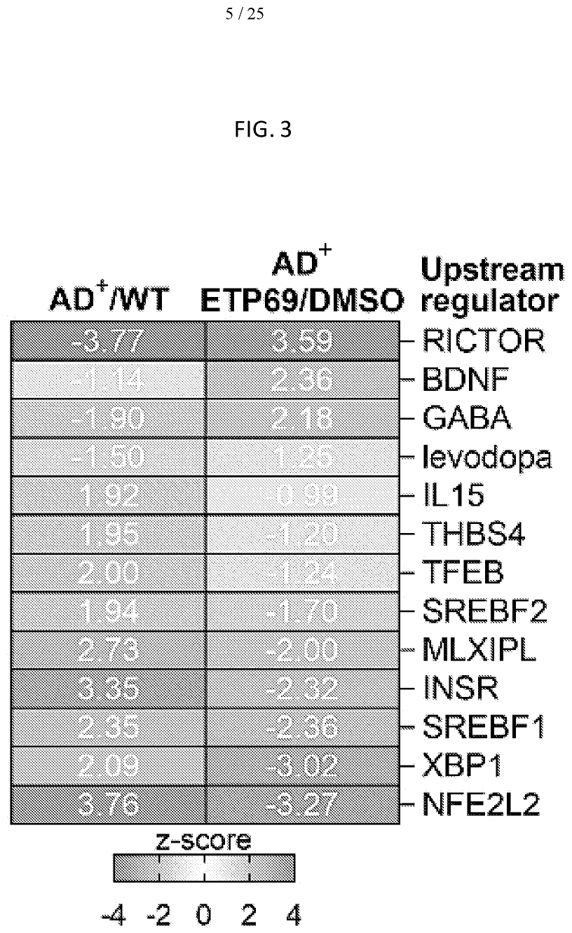

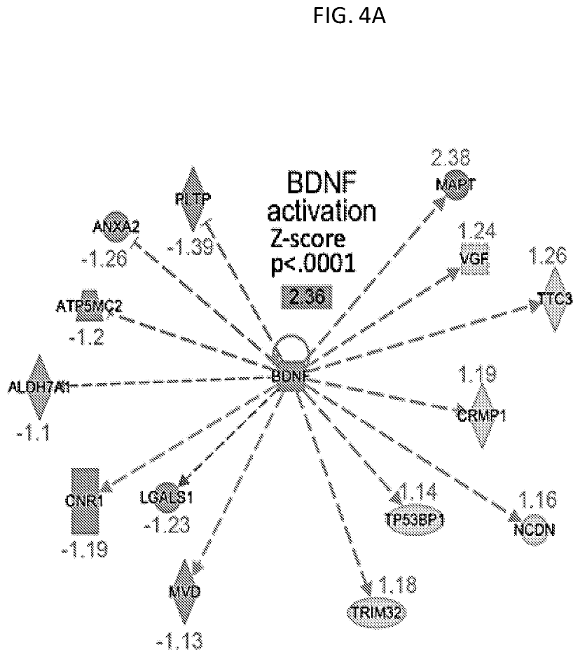

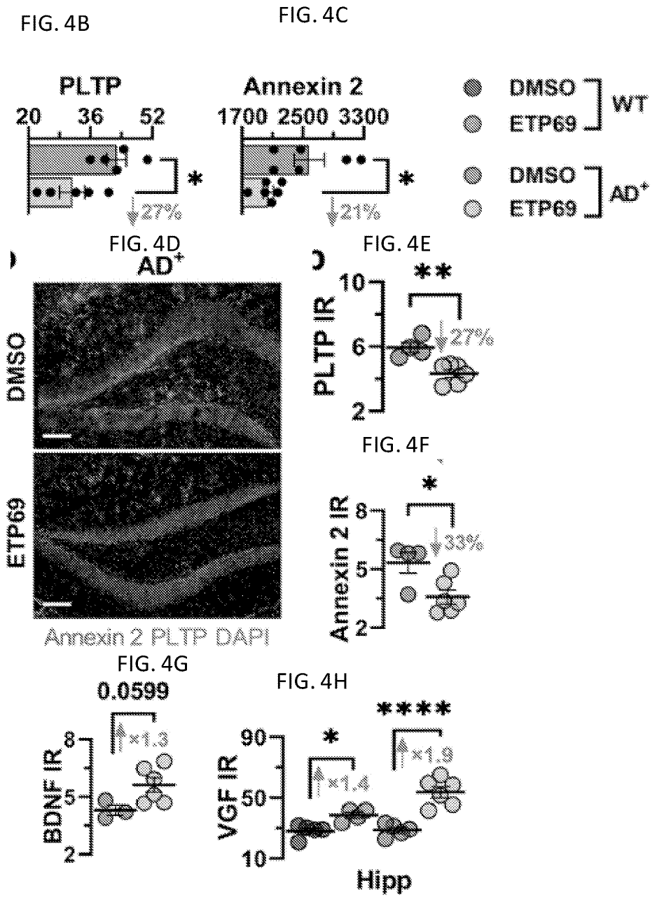

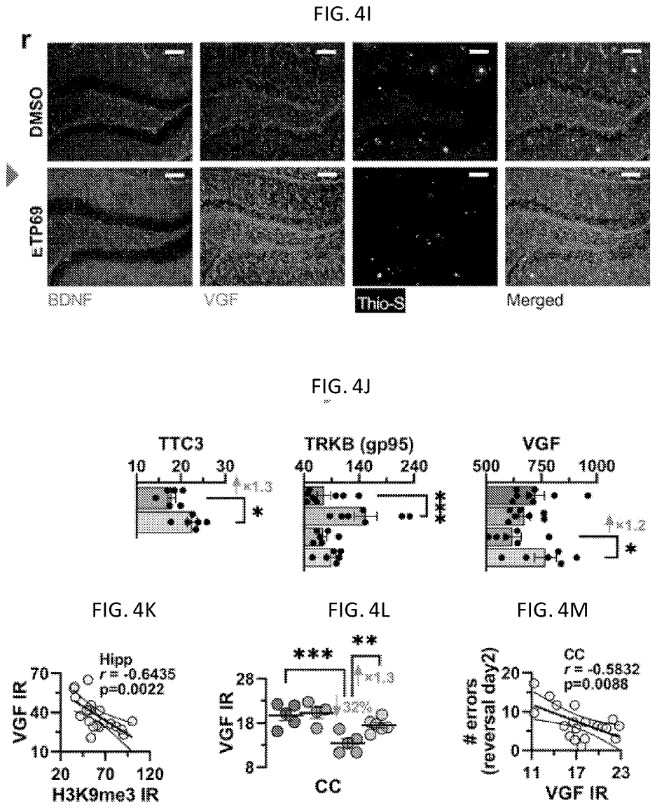

Gene expression was measured in mice treated with ETP69 and DMSO. As depicted in FIG. 3, genes in the BDNF and VGF pathways were activated. These pathways are consistent with protective neurotrophic support. This pathway correlates with reduced H3K9me3 and improved cognition. FIG. 4A depicts genes in the BDNF pathway inhibited (left) and activated (right) by ETP69. The inactivated genes are quantified in FIGS. 4B-4F. The activated genes are quantified in FIGS. 4G-4M.

### Example 4: Treatment of Mice with ETP69

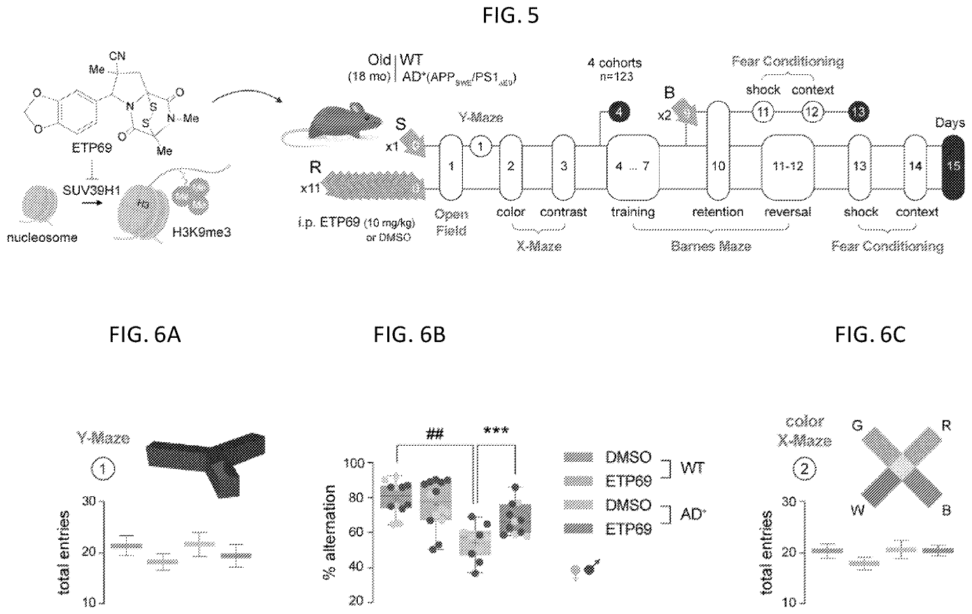

The effects of single and repeated ETP69 injections in mice were tested. The experimental timeline is depicted in FIG. 5. Three parallel injection conditions were tested: a single ETP69 condition, a boosted ETP69 injection, and repeated ETP69 injections. The single injection mice received one injection of ETP69 or DMSO at Day 0. The mice that received a boosted injection received one injection of ETP69 or DMSO at day 0 and one injection at day 9. The mice that received repeated injections received 11 injections of ETP69 or DMSO at day 0 or day 9. Both wildtype and AD model (APP/PS1-transgenic) mice were tested.

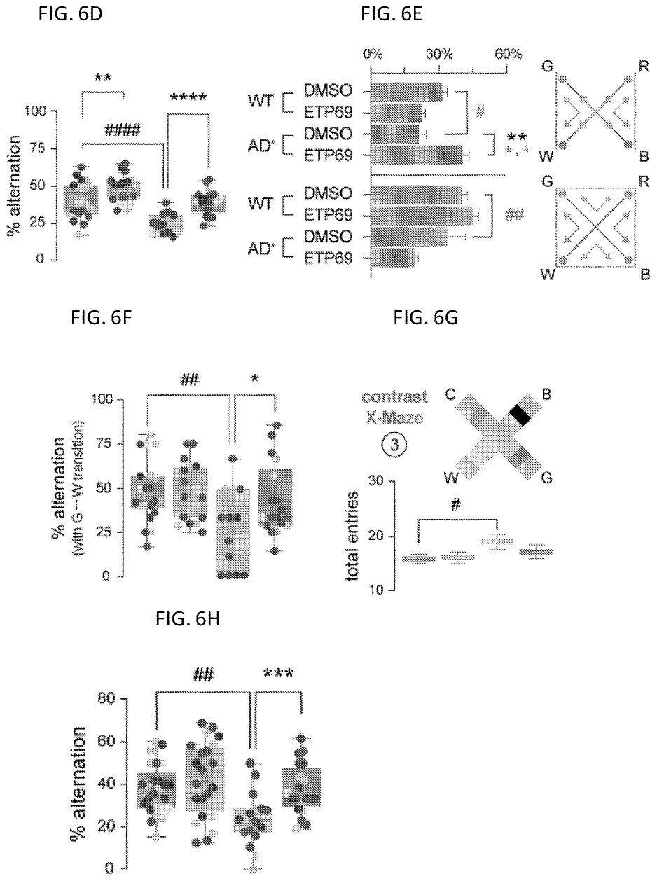

Treatment with ETP69 restored CNS-related locomotor, visual, cognitive functions (FIGS. 6A-611). First, the effects of ETP69 on cognitive and visual protection was tested using the Y-Maze test. Both wildtype and AD+ mice treated with ETP69 showed a decreased number of total entries (FIG. 6A), indicating decreased locomotor activity. When looking at the percent alternations, a proxy for cognition, AD+ mice administered DMSO showed a significant decrease compared to wildtype mice administered DMSO. However, when AD+ mice were treated with ETP69, there was a significant increase in the percent alternation (FIG. 6B).

FIG. 6C-6F depicts the results of the mice on the color mode of the visual stimuli X-maze test. When looking at the percent alternations, a proxy for cognition, AD+ mice administered DMSO showed a significant decrease compared to wildtype mice administered DMSO. However, when AD+ mice were treated with ETP69, there was a significant increase in the percent alternation and bidirectional movement (FIG. 6C-6F). FIG. 6G-611 depict the results of the contrast mode of the visual stimuli X-maze test. When looking at the percent alternations, AD+ mice administered DMSO showed a significant decrease compared to wildtype mice administered DMSO. However, when AD+ mice were treated with ETP69, there was a significant increase in the percent alternation.

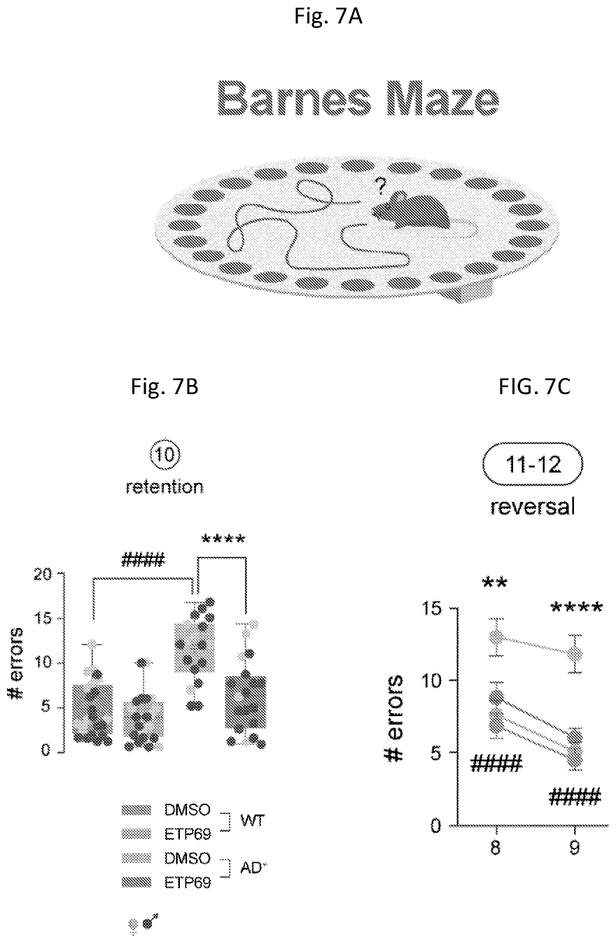

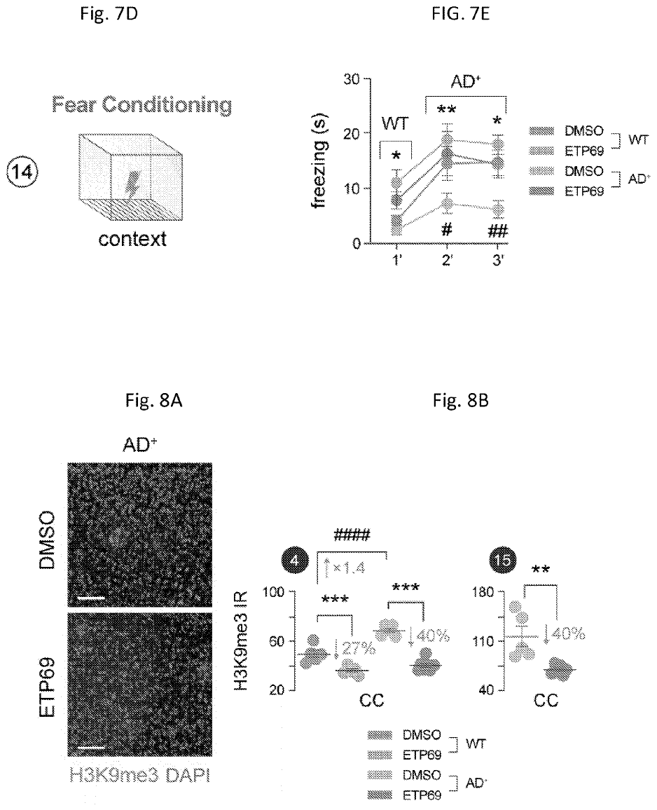

ETP69 treatment restored cognitive-behavioral functions (FIGS. 7A-7E). The results of the Open Field Barnes Maze test indicate hippocampal-based spatial memory. FIG. 7A depicts a schematic of the Barnes Maze Test. AD+ mice treated with ETP69 showed an improvement in memory retention compared to AD+ mice treated with DMSO (FIG. 7A-7C). The results of the fear conditioning test indicate spatial memory. AD+ mice treated with ETP69 showed increased freezing time, indicating memory protection (FIG. 7D-7E).

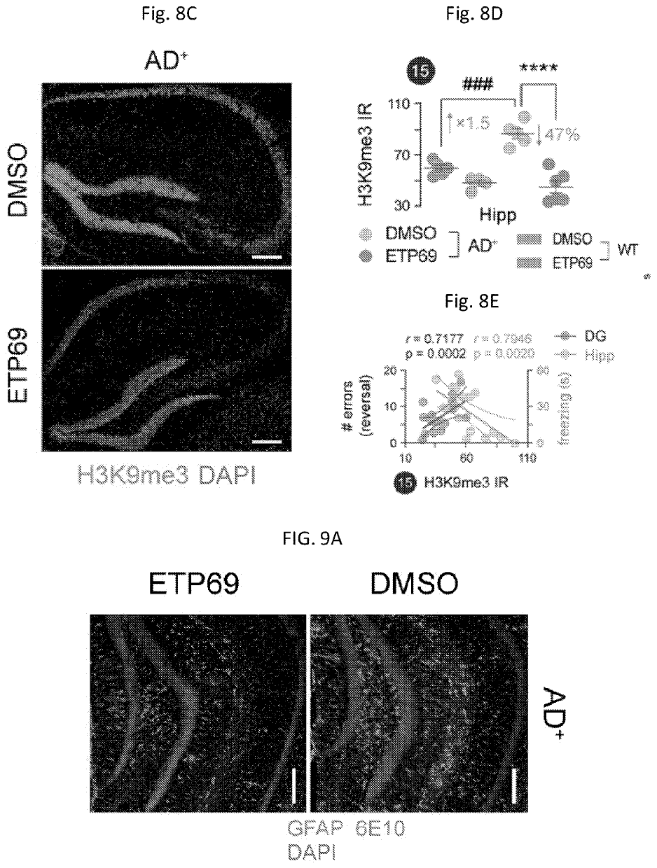

ETP69 treatment resulted in reduced epigenetic H3K9me3 mark repression (FIGS. 8A-8E). AD+ brains were stained for neuronal H3K9me3 and DAPI. Representative images of the cingulate cortex and hippocampus of DMSO and ETP69 treated mice are depicted in FIGS. 8A and 8C, respectively. Neuronal H3K9me3 irradiance in the cingulate cortex is quantified in FIG. 8B. Both wildtype and AD+ mice administered ETP69 showed a decreased in H3K9me3 irradiance (27% and 40%, respectively) at day 4. AD+ mice administered ETP69 showed a decrease in H3K9me3 irradiance of 40% at day 14. In the hippocampus, AD+ mice administered DMSO showed a 47% decrease in irradiance compared to AD+ mice administered DMSO (FIG. 8E). H3K9me3 staining in the DG correlated with performance in the Barnes Test, while K3K9me3 staining in the hippocampus was correlated with performance in the fear conditioning test.

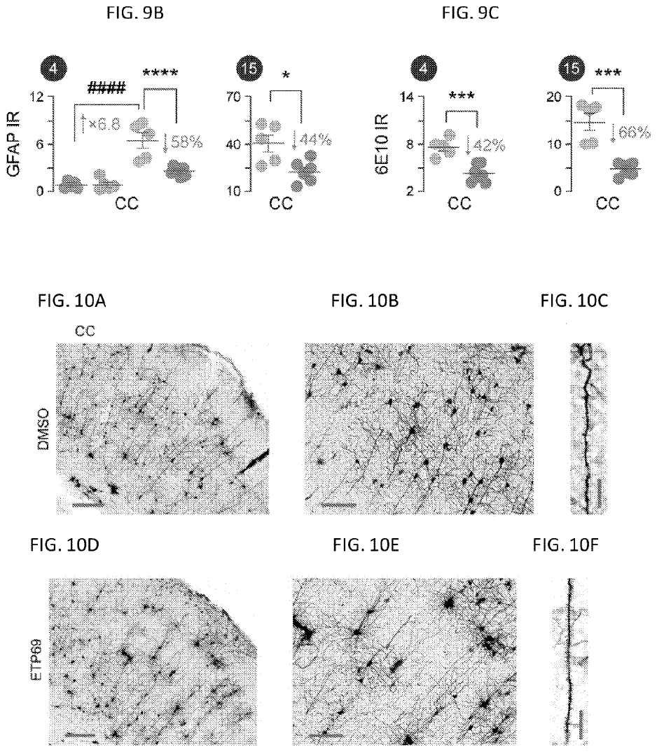

ETP69 administration reduced AD-related brain pathology (FIGS. 9A-9C). Brain sections were stained for H3K9me3, 6E10 and GFAP. Representative images are shown in FIG. 9A. AD+ mice showed a 6.8 times increase in levels of GFAP compared to wildtype mice administered DMSO (FIG. 9B). Administration of ETP69 resulted in a significant decrease of GFAP and 6E10 at both 4 and 15 days in AD+ mice (FIGS. 9B-9C).

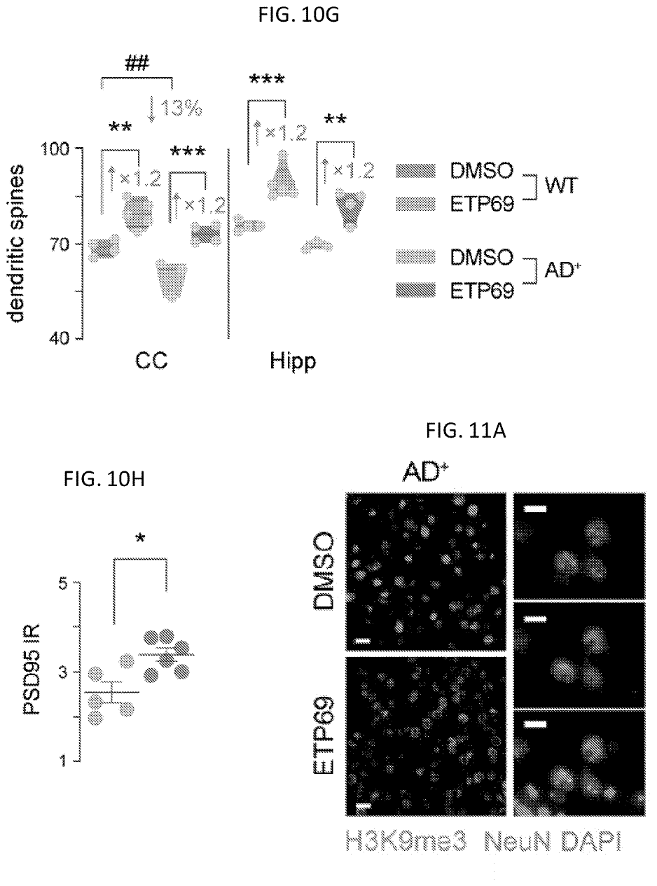

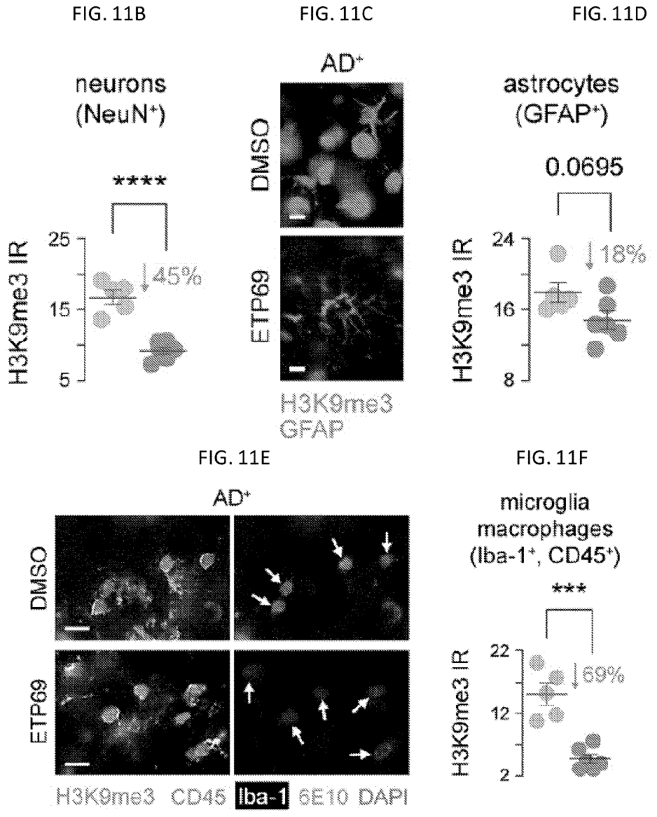

The brains were stained with the Golgi-Cox method to detect the cytoarchitecture and morphology of neurons. Representative images are shown of mice treated with DMSO (FIG. 10A-10C) and ETP69 (FIG. 10D-10F). The number of dendritic spines are quantified. ETP69 administration significantly increased the number of dendritic spines in both the cingulate cortex and the hippocampus (FIG. 10G). ETP69 administration also resulted in increased expression of PSD95 irradiance (FIG. 10H). Further, ETP69 administration reduced epigenetic H3K9me3 repression mark in cell-specific manners. H3K9 expression was reduced in neurons (FIGS. 11A-11B), astrocytes (FIGS. 11C-11D), and microglia macrophages (FIGS. 11E-11F).

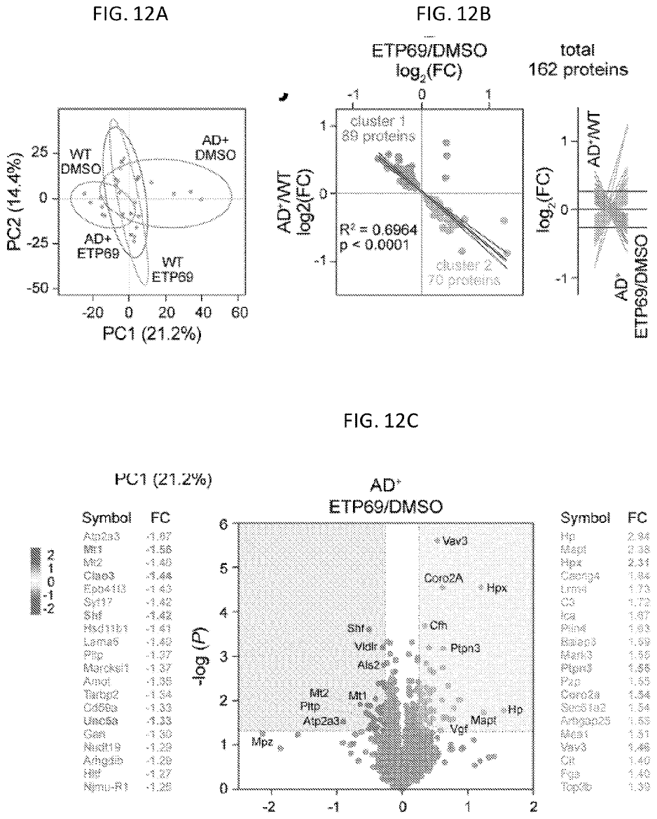

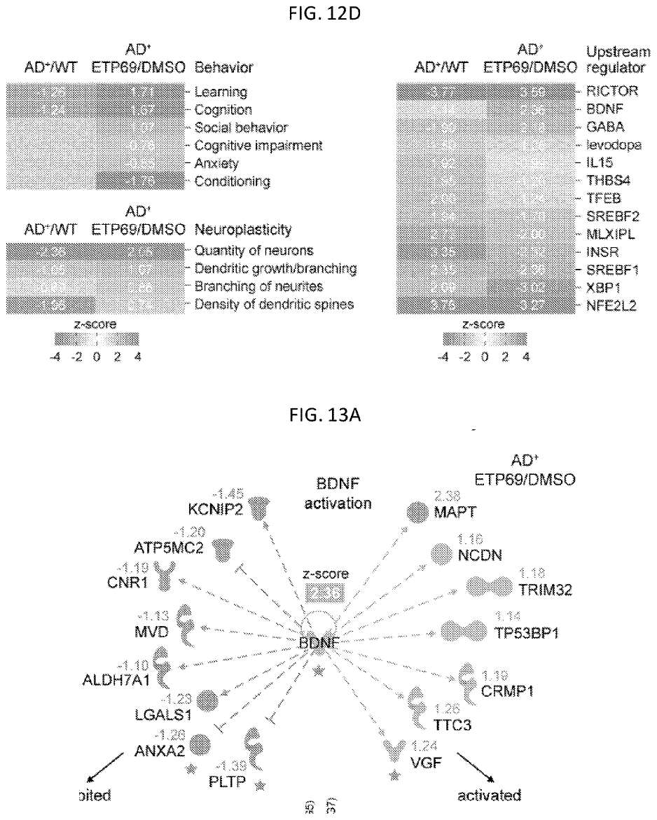

Proteome signatures revealed that ETP69 induced proteostasis along with increased learning and memory-enriched pathways and NTFs (FIGS. 12A-12D). The results included levodopa (L-dopa), a naturally occurring product that is also a drug for the management of Parkinson's motor symptoms.

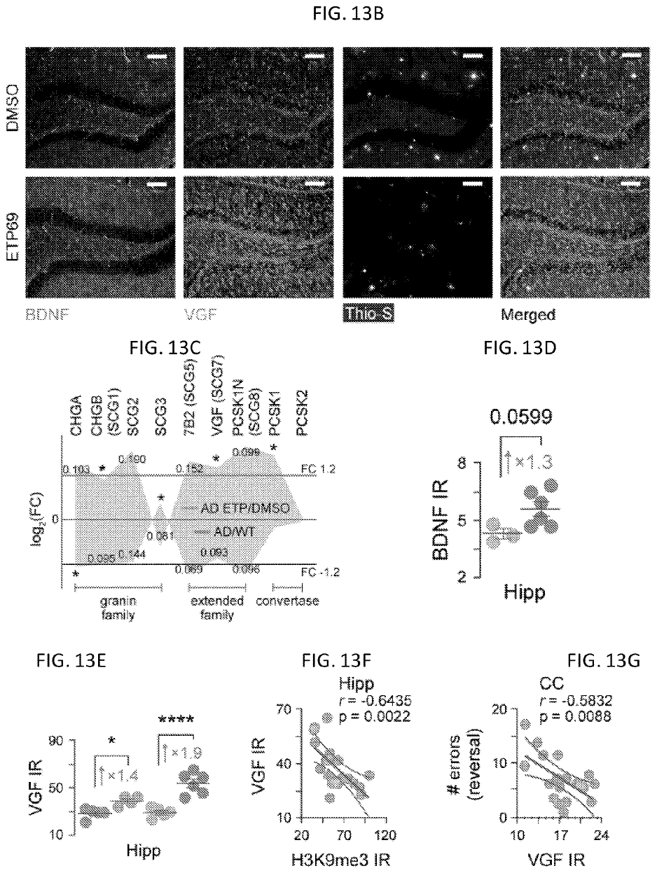

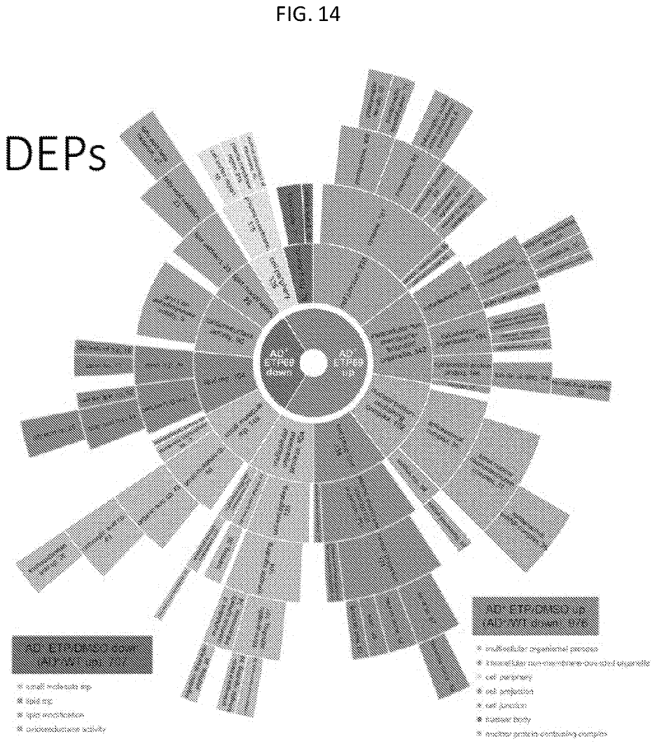

Further, the proteosome signatures that ETP69 induced brain neutrotrophic factors (NTFs) expression including BDNF, VGF and levodopa (FIG. 13A-13G). There was enrichment in spine and dendritic preservation pathways in the differentially expressed proteins (FIG. 14)

### Example 5: ETP69 Administration is Neuroprotective in Old Mice in Both a Neurodegenerative Mouse Model and in Wildtype Mice

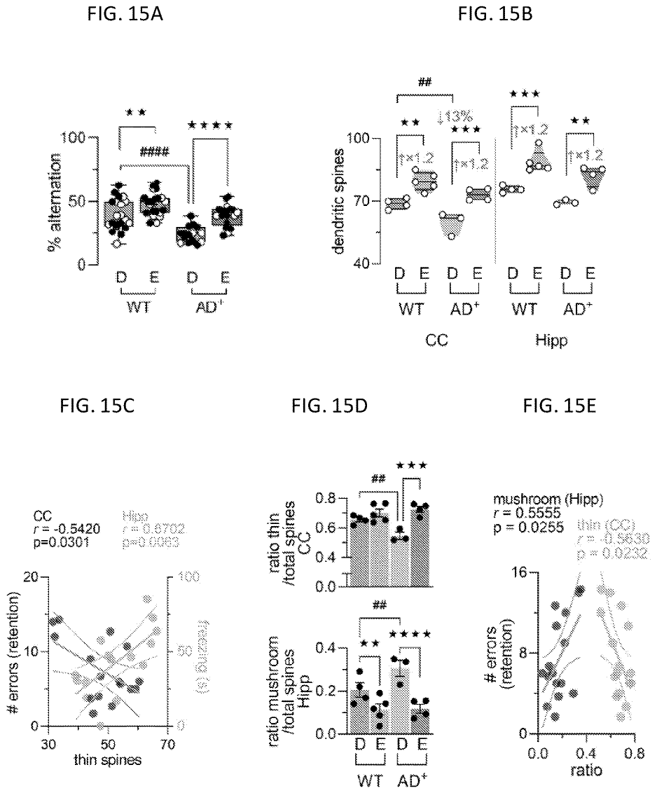

Old (18-month-old) mice were administered ETP69. Both a neurodegenerative model (AD+) and wild type siblings were tested. Mice were treated with i.p. injection of ETP69 [E; 10 mg/kg dissolved in dimethyl sulfoxide (DMSO)]. A series of behavioral tests were performed to assess locomotor, cognitive and visual functions. a single dose of ETP69 also increased the colour X-maze spontaneous alternations in old WT mice (p=0.0027; FIG. 15A), indicating that ETP69 can improve cognitive function in normal ageing.

Loss of synapses and dendritic spine abnormalities are associated with cognitive decline. To analyze these features in old mice administered ETP69, Golgi-Cox neuronal staining and synaptic marker staining was performed in cerebral cortices and hippocampi. Dendritic spines were classified as thin (filopodia-like and long-thin), stubby, or mushroom spines according to their size and shape, and quantified the spine density from high-magnification photographs.

Both wildtype mice and neurodegenerative mice showed a 1.2 fold increase in the number of dendritic spines in both the hippocampus and the cerebral cortex of mice administered ETP69 compared to mice administered a DMSO control (FIG. 15B).

There was also a functional correlation between the increase in density of the dendritic spines and improved cognition. Thin spines are newly formed and associated with new learning and memory formation. Lower thin spine density in the cortex and hippocampus was correlated with poorer performance on the Barnes maze test and contextual fear conditioning test (r=−0.54, p=0.03 and r=0.67, p=0.0063, respectively; FIG. 15C). Analysis of dendritic spine ratio showed that ETP69 treatment induced higher thin-to-total spine ratio and lower mushroom-to-total spine ratio (FIG. 15D), counteracting the effects of AD on these dendritic spine subtypes. Moreover, improved cognitive performance on the Barnes maze test was correlated with higher thin- and lower mushroom-to-total spine ratios (FIG. 15E).

### Example 6: ETP69 Administration Reduces Repressive Marks in the Cerebral Cortex

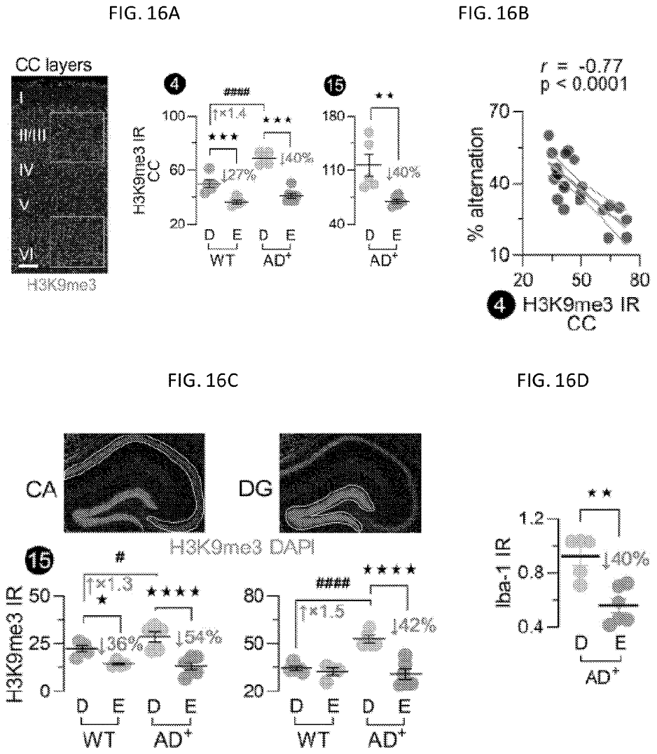

This example shows how ETP69 may reduce repressive marks, an affect that may be associated with anti-degeneration. H3K9me3 immunoreactive area was distributed in all the layers of the cerebral cortex but showed differences between layers due to variable neuronal population density. Quantification was therefore performed in the most densely populated cortical layers II/III and VI, as shown in FIG. 16A. The cortical and hippocampal levels of H3K9me3 were significantly 1.3-1.5-fold increased in DMSO-control neurodegenerative (AD+) compared to WT mice (p<0.05-0.0001; FIG. 2h-l). Elevated cortical H3K9me3 significantly and negatively correlated with cognitive performance, as observed on the X-maze color mode (r=−0.77, p<0.0001; FIG. 16B).

The effects of ETP69 on cerebral H3K9me3 levels was tested in old mice, in both a neurodegenerative model and WT siblings. The levels of brain H3K9me3 at days 4 (short-term) and 15 (long-term) following i.p. ETP69 injection (S regimen cohorts) were measured. The data indicated that ETP69 dramatically reduce H3K9me3 IR area in the cortex of both WT (27%, p=0.0004) and neurodegenerative (40%, p<0.0001) old mice at 4 days after the injection. Notably, the magnitude of cortical H3K9me3 reduction by ETP69 remained stable even after 15 days in neurodegenerative mice (40%, p=0.0069; FIG. 16A). Quantitative IHC analysis showed that ETP69 treatment persistently reduced hippocampal H3K9me3 levels 15 days after injection in the WT mice (CA, 36%, p<0.05; FIG. 2I) and in the neurodegenerative mice (CA, 54%, p<0.0001; DG, 42%, p<0.0001; FIG. 16C). These findings indicate that a single dose of ETP69 is sufficient to sustainably reduce H3K9me3 in the brains of old WT and neurodegenerative mice. Consistent with the observations in 18-month-old mice, ETP69 mitigated neuroinflammation, including by reduced ionized calcium-binding adaptor molecule 1 (Iba-1)⁺ microgliosis in neurodegenerative mice (40%, p=0.0021; FIG. 16D).

### Example 7: ETP69 Administration Activates Pathways Associated with Learning and Cognition

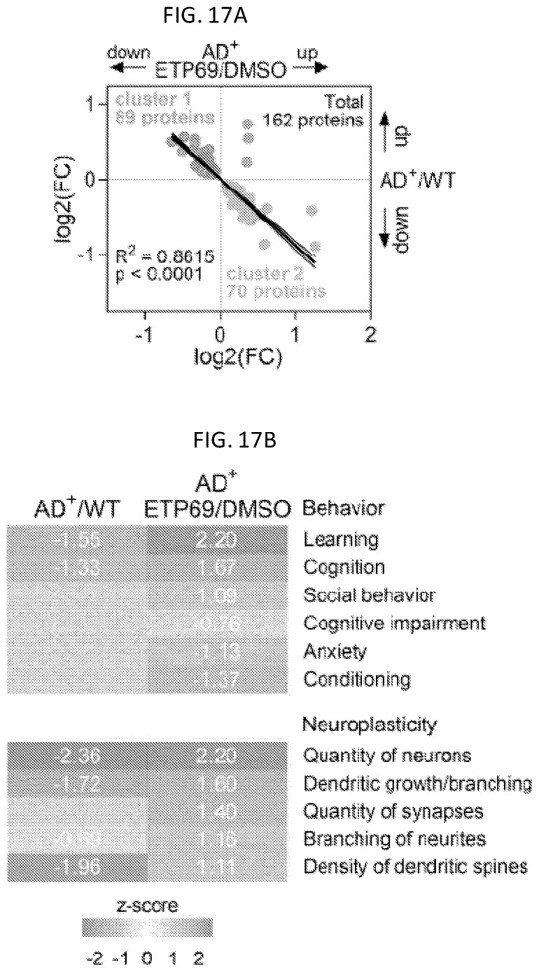

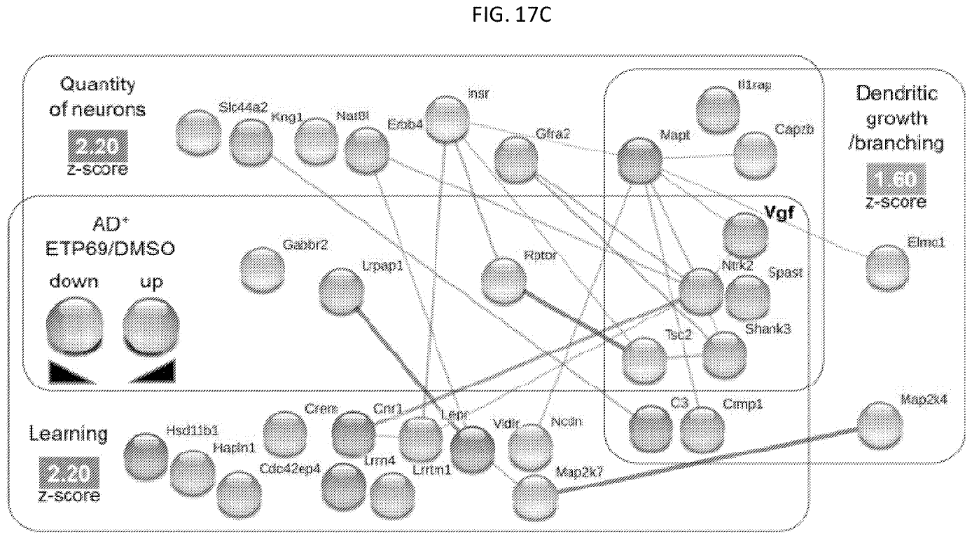

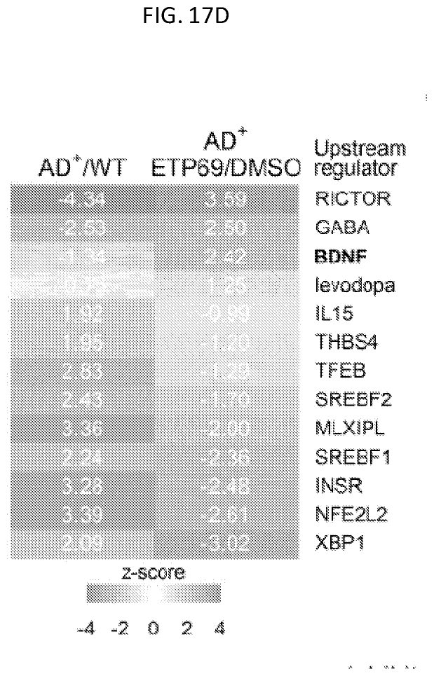

Mass spectrometry (MS) analysis on the soluble fraction of brain homogenates extracted from the 14-month-old WT and neurodegenerative (AD) mice cohort was performed (DMSO-treated WT mice: n=8; ETP69-treated WT mice: n=7; DMSO-treated AD⁺ mice: n=6; ETP69-treated AD⁺ n=6). Of the 162 overlapping DEPs between the AD⁺/WT and AD⁺ ETP69/DMSO comparisons, 159 DEPs that were either up- or downregulated due to disease state (AD⁺ vs. WT) were reversed by ETP69 treatment (R²=0.86, p<0.0001; FIG. 17A).

The effects of ETP69 on the molecular pathways related to neuroprotection and cognitive function was assessed. Ingenuity Pathway Analysis (IPA) revealed that ETP69 administration significantly activated pathways of learning, cognition, quantity of neurons, as well as dendritic growth and branching, fully reversing the AD-related inhibitory effects of these pathways (FIG. 17B). A STRING network shows the overlapping DEPs between the three functions: learning, quantity of neurons, and dendritic growth/branching (FIG. 17C). The data indicated that the leucine-rich repeat neuronal protein 4 (Lrrn4), which plays an important role in hippocampus-dependent learning and long-lasting memory, was a top 1.73-fold upregulated protein in ETP69-treated versus DMSO-control AD⁺ mice (p=0.015). Further, the glial cell line-derived neurotrophic factor (GDNF) receptor 2 (Gfra2) that promotes neuronal survival, was a top downregulated protein in AD⁺ mice and significantly increased in ETP69-treated mice (p=0.0056). Interestingly, the microtubule-associated protein tau (Mapt), a major constituent of neuronal cytoskeleton, was also a top downregulated protein in AD⁺ versus WT mice and top 2.4-fold upregulated in response ETP69 in AD⁺ mice (p=0.0188).

The five proteins that overlapped between the three pathways included the Bdnf/Nt-3 growth factor receptor (neurotrophic receptor tyrosine kinase 2; Ntrk2), involved in neuron survival and neuroplasticity, the SH3 and multiple ankyrin repeat domains protein 3 (Shank3), a major scaffold postsynaptic density protein, spastin (Spast) involved in axon growth, the tuberous sclerosis complex 2 (Tsc2, also known as tuberin), and the neurosecretory protein nerve growth factor-inducible Vgf involved in neurogenesis and neuroplasticity (FIG. 6b).

The top IPA upstream regulators that were induced or inhibited by ETP69 treatment are depicted in FIG. 17C. Rictor, GABA, and BDNF were top activated networks in response to ETP69 treatment (z scores: 3.59, 2.50, 2.42, respectively), whereas these pathways were among the top inhibited pathways in AD⁺ versus WT mice.

### Example 8: A Clinical Trial for the Treatment of Parkinson's Disease

The purpose of this study is to study the effect of ETP69 on Parkinson's disease. In addition, this study will generate data on the safety, tolerability, and pharmacokinetics of the treatment in subjects with Parkinson's disease. Individuals will be administered placebo or ETP69 weekly and monitored for at least 12 weeks of study. Subjects will be assessed for motor and cognitive measurements before the trial and at the conclusion of the treatment. The treatment may ameliorate at least one symptom of Parkinson's disease. The treatment may delay the progression of at least one symptom of Parkinson's disease.

### Example 9: A Clinical Trial for the Treatment of Huntington's Disease

The purpose of this study is to study the effect of ETP69 on Huntington's disease. In addition, this study will generate data on the safety, tolerability, and pharmacokinetics of the treatment in subjects with Huntington's disease. Individuals will be administered placebo or ETP69 weekly and monitored for at least 12 weeks of study. Subjects will be assessed for motor and cognitive measurements before the trial and at the conclusion of the treatment. The treatment may ameliorate at least one symptom of Huntington's disease. The treatment may delay the progression of at least one symptom of Huntington's disease.

### Example 10: A Clinical Trial for the Treatment of Amyotrophic Lateral Sclerosis (ALS)

The purpose of this study is to study the effect of ETP69 on ALS. In addition, this study will generate data on the safety, tolerability, and pharmacokinetics of the treatment in subjects with ALS. Individuals will be administered placebo or ETP69 weekly and monitored for at least 12 weeks of study. Subjects will be assessed for motor and cognitive measurements before the trial and at the conclusion of the treatment. The treatment may ameliorate at least one symptom of ALS. The treatment may delay the progression of at least one symptom of ALS.

### Example 11: A Clinical Trial for the Treatment of Learning Disorders

The purpose of this study is to study the effect of ETP69 on learning disorders. In addition, this study will generate data on the safety, tolerability, and pharmacokinetics of the treatment in subjects with learning disorders. Individuals will be administered placebo or ETP69 weekly and monitored for at least 12 weeks of study. Subjects will be assessed for motor and cognitive measurements before the trial and at the conclusion of the treatment. The treatment may ameliorate at least one symptom of a learning disorder.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of treating Parkinson's disease, Huntington's disease, ALS, or a learning disorder in a subject in need thereof, the method comprising administering to the subject a compound comprising an inhibitor of trimethylation of histone 3, lysine 9 (H3K9).

2. The method of claim 1, wherein the compound comprises an inhibitor of SUV39H1.

3. The method of claim 1, wherein the compound comprises an inhibitor of SUV39H2.

4. The method of claim 1, wherein the compound has the structure of Formula (I), or a pharmaceutically acceptable salt thereof: 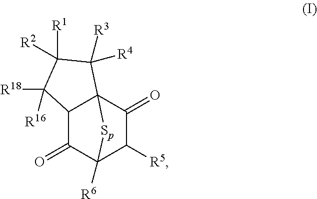
wherein, p is 2, 3 or 4, and
R¹, R², R³, R⁴, R⁵, R⁶, R¹⁶, and R¹⁸ are independently hydrogen, halogen, —N₃, —CF₃, —CCl₃, —CBr₃, —Cl₃, —CN, —CHO, —OH, —NH₂, —COOH, —CONH₂, —NO₂, —SH, —SO₂, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

5. The method of claim 1, wherein the compound comprises (Rac-(3S,6S,7S,8aS)-6-(benzo[d][1,3]dioxol-5-yl)-2,3,7-trimethyl-1,4-dioxohexahydro-6H-3, 8a-epidithiopyrrolo[1,2-a]pyrazine-7-carbonitrile) (ETP69), or a pharmaceutically acceptable salt thereof.

6. The method of claim 1, wherein the administration improves locomotor activity in the subject.

7. The method of claim 1, wherein the administration reduces neuroinflammation in the subject.

8. The method of claim 1, wherein the administration increases or improves a neuroprotective phenotype.

9. The method of claim 7, wherein the administration activates brain-derived neurotrophic factor (BDNF) or VGF nerve growth factor inducible (VGF) signaling in the subject.

10. The method of claim 1, wherein the administration reduces or improves a phenotype associated with Parkinson's disease, Huntington's disease, ALS, or the learning disorder in the subject.

11. The method of claim 10, wherein the subject has or has been diagnosed with Parkinson's disease.

12. The method of claim 10, wherein the subject has or has been diagnosed with Huntington's disease.

13. The method of claim 10, wherein the subject has or has been diagnosed with ALS.

14. The method of claim 10, wherein the subject has or has been diagnosed with the learning disorder.

15. A method of inhibiting an enzyme selected from the group consisting of: ASH1L, MLL1 complex, MLL4 complex, NSD3, Set1b complex, SMYD2, SMYD3, SUV39H1, and SUV39H2, comprising contacting the enzyme with a compound having the structure of Formula (I), or a pharmaceutically acceptable salt thereof: 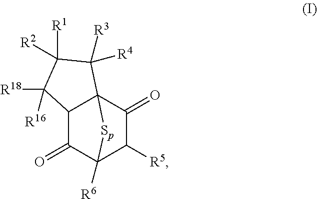
wherein, p is 2, 3 or 4, and
R¹, R², R³, R⁴, R⁵, R⁶, R¹⁶, and R¹⁸ are independently hydrogen, halogen, —N₃, —CF₃, —CCl₃, —CBr₃, —Cl₃, —CN, —CHO, —OH, —NH₂, —COOH, —CONH₂, —NO₂, —SH, —SO₂, —SO₂Cl, —SO₃H, —SO₄H, —SO₂NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

16. The method of claim 15, wherein the compound comprises (Rac-(3S,6S,7S,8aS)-6-(benzo[d][1,3]dioxol-5-yl)-2,3,7-trimethyl-1,4-dioxohexahydro-6H-3, 8a-epidithiopyrrolo[1,2-a]pyrazine-7-carbonitrile) (ETP69), or a pharmaceutically acceptable salt thereof.

17. The method of claim 15, wherein the contact is in vitro.

18. The method of claim 15, wherein the contact is in vivo.

19. The method of claim 18, wherein the contact is in a neuron of a subject.

20. The method of claim 18, wherein the contact is in a human subject.

21. The method of claim 15, further comprising assessing an activity of the enzyme during or after the contact of the enzyme with the compound.

22. The method of claim 21, wherein the activity of the enzyme is decreased by at least 75% after the contact of the enzyme with the compound.
